(19) United States
(12) Patent Application Publication
(10) Pub. No.: US 2003/0124565 A1
(43) Pub. Date: Jul. 3, 2003

(54) Efficient methods for assessing and validating ecandidate protein-based therapeutic molecules encoded by nucleic acid sequences of interest

(76) Inventors: Leonard I. Garfinkel, Northrige, CA (US); Andrew L. Pearlman, Misgav (IL); Eduardo N. Mitrani, Jerusalem (IL)

Correspondence Address: G.E. EHRLICH (1995) LTD., c/o ANTHONY CASTORINA, 2001 JEFFERSON DAVIS HIGHWAY, SUITE 207, ARLINGTON, VA, 22202, US

(21) Appl. No.: 10/190,754

(22) Filed: Jul. 9, 2002

Related U.S. Application Data
(60) Provisional application No. 60/303,337, filed on Jul. 9, 2001.

(51) Int. Cl. C12Q 1/68; A61K 49/00

(52) U.S. Cl. 435/6; 424/9.2

(57) ABSTRACT

A method of determining at least one quantitative or qualitative pharmacological, physiological and/or therapeutic, parameter or effect of a recombinant gene product in vivo, the method comprises (a) obtaining at least one micro-organ explant from a donor subject, the micro-organ explant comprising a population of cells, the micro-organ explant maintaining a microarchitecture of an organ from which it is derived and at the same time having dimensions selected so as to allow diffusion of adequate nutrients and gases to cells in the micro-organ explant and diffusion of cellular waste out of the micro-organ explant so as to minimize cellular toxicity and concomitant death due to insufficient nutrition and accumulation of the waste in the micro-organ explant, at least some cells of the population of cells of the micro-organ explant expressing and secreting at least one recombinant gene product; (b) implanting the at least one micro-organ explant in a recipient subject; and (c) determining the at least one quantitative or qualitative pharmacological, physiological and/or therapeutic, parameter or effect of the recombinant gene product in the recipient subject.

This application is a continuation of PCT/IL02/XXXXX, filed Jul. 7, 2002, having the same title and identified by Attorney Docket No. 02/23844, which claims the benefit of priority from U.S. Provisional Patent Application No. 60/303,337, filed Jul. 9, 2001.

### FIELD AND BACKGROUND OF THE INVENTION

The present invention relates to methods of rapid assessment and validation of candidate protein-based therapeutic molecules encoded by nucleic acid sequences of interest. The present invention also relates to methods of determining at least one quantitative or qualitative pharmacological, physiological and/or therapeutic parameter or effect of an expressed recombinant gene product in vitro or in vivo. More particularly, the present invention relates to a method of determining these effects in an in vivo system utilizing micro-organs as a means of expressing nucleic acids of interest.

The human genome project has provided the scientific world and the biotechnological and pharmaceutical industries with an enormous amount of data regarding new genes, ESTs (expressed sequence tags) and SNPs (single nucleotide polymorphisms) which encode novel or modified proteins. These putative proteins are potential candidates for the development of new protein-based therapies for human and veterinary diseases.

During the process of protein-based drug discovery, specific protein molecules are identified as potential protein-based drugs. The interaction between a particular protein-based drug and its cellular target in vivo should be assessed at the earliest possible stage of the drug development process, prior to proceeding with the development of a lead compound for a specific disease. Drug validation has become an essential requirement for the design of protein-based drugs and assists in deciding whether or not critical resources will be expended on a candidate drug. From this point of view, it is just as important to invalidate a protein-based drug, which does not show sufficient physiological/therapeutic effect.

Currently, a variety of in vitro approaches exist for rapid profiling of either promising nucleic acid sequences or their corresponding proteins, which may be active in numerous biological and disease processes. These approaches can help determine gene/protein function, its direct or regulatory role in a disease state and its potential as a therapeutic protein. However, in vitro study can give only limited information, and animal-based systems must be used to reach operative conclusions regarding the biological/physiological effect/activity of the protein or nucleic acid sequence. However, current in vivo approaches require lengthy and expensive procedures for protein production, purification and formulation, all before administration to an animal is even possible. For example, an animal model, whether wild type or a disease model, may be exposed to a protein suspected of exhibiting an ability to interact with a given receptor (e.g., receptor agonist), stimulating a regulatory cascade, providing missing enzymatic activity, etc. Monitoring animal responses to the administration of such a protein can be accomplished by assessing the extent of change in response to exposure to the protein, and associated physiological effects.

Existing protein production techniques involve the sub-cloning of a desired nucleic acid sequence/fragment into a vector, typically a plasmid, phage or virus. Such a recombinant vector is subsequently used for transducing specific host cells, which will produce the desired protein for further purification steps. Such host cells are well known in the art and include, for example: bacterial cells, yeast cells, insect cell cultures, mammalian tissue cultures and plant cells. It is often difficult, time consuming, costly, and sometimes even impossible to achieve high-level expression of a given recombinant protein. Each of the above-described hosts has limitations in terms of either the amount of protein expressed, or other aspects of the protein, which relate to its activity in the intended use. For example, proteins expressed in bacterial cells, which are the easiest to manipulate, are often maintained in a non-secreted manner inside the bacterial cell and more specifically are localized within inclusion bodies from which it is oftentimes difficult to isolate and purify them. Furthermore, a bacterial cell cannot provide to the protein many of the post-translational modifications (such as glycosylation and the accurate folding of the protein) that may be required for its biological activity. On the other hand, eukaryotic protein production systems may result in inaccurate post-translational modification. In certain circumstances, an expressed recombinant protein might be toxic to the host cells, which further prevents production of reasonable amounts for assessing that protein.

Moreover, even after a high-level of protein production has been achieved, large quantities of the recombinant protein must then be produced and purified to be free of contaminants. Development of a purification scheme is a very lengthy process. Often, it is necessary to sustain substantial production losses with very low yields in order to obtain recombinant protein of the necessary purity.

Once purified recombinant protein has been obtained, it must be further formulated to render it stable and acceptable for introduction into animals or humans. The process of developing an appropriate formulation is time consuming, difficult, and costly, as well.

Furthermore, even formulated, purified recombinant proteins have a finite shelf life due to maintenance and storage limitations; often requiring repeated purification and formulation of more protein. Batch-to-batch variation encountered in such an approach may complicate the data obtained in animal studies using these proteins.

All the above-described protein production techniques are very lengthy and costly, and frequently do not yield sufficient, biologically active amounts of the desired protein to enable the intended required analysis in vitro and in vivo.

Thus, there is a widely recognized need for a method for assessing and validating the biological activity of candidate nucleic acid sequences encoding protein-based therapeutic molecules, without the need for the aforementioned production steps. Furthermore, there is a need for a method for increasing the likelihood that the protein thus produced will have the requisite post-translational modifications to preserve its biological activity.

In particular, there is a widely recognized need for, and it would be highly advantageous to have, a method of evaluating potential protein-drug candidates in an in vivo setting. Methods enabling in vivo expression of recombinant gene products circumventing the laborious and costly methods typically associated with obtaining high-levels of recombinant proteins, as outlined above, are clearly advantageous. Methods providing for in vivo expression of recombinant gene products that require post-translational modifications, or are toxic to host cells typically used in these applications, are of primary importance.

An alternative prior art method enabling in vivo expression of recombinant gene products is gene therapy. Typically viral vectors are used to transduce via transfection cells in vivo to express recombinant gene products. These viral-based vectors have advantageous characteristics, such as the natural ability to infect the target tissue. However, several as yet insurmountable limitations plague their efficient application. Retrovirus-based vectors require integration within the genome of the target tissue to allow for recombinant product expression (with the potential to activate resident oncogenes) while vector titers produced in such systems are not exceptionally high. Additionally, because of the requirement for retroviral integration within the subject's genome, the vector can only be used to transduce actively dividing tissues. Further, many retroviruses have limited host tissue specificity and cannot be employed to transduce more than a few specific tissues of the subject.

Other DNA based viral vectors suffer limitations as well, in terms of their inability to sustain long-term transgene expression; secondary to host immune responses that eliminate virally transduced cells in immune-competent animals (Gilgenkrantz et al., Hum. Gene Ther. 6:1265 (1995); Yang et al., J. Virol. 69:2004 (1995); Yang et al., Proc. Natl. Acad. Sci. USA 91:4407 (1994); and Yang et al., J. Immunol. 155: 2565 (1995)). While immune responses were directed against the transgene-encoded protein product (Tripathy et al., Nat. Med. 2; 545-550 (1996)), vector epitopes were a trigger for host immune responses, as well (Gilgenkrantz et al., Hum. Gene Ther. 6:1265 (1995); and Yang et al., J. Virol. 70: 7209 (1996)).

These combined limitations result in inconsistent recombinant gene product expression, and a difficulty in determining accurate expression levels of the recombinant product, and little opportunity for prolonged in vivo expression. Accordingly, there remains a need in the art for improved systems for generating recombinant gene products that address these limitations.

### SUMMARY OF THE INVENTION

The present invention discloses the utilization of recombinant gene products expressed in genetically modified micro-organs for the determination of pharmacological, physiological and/or therapeutic, quantitative or qualitative parameters or effects in experimental in vivo models. Genetically modified micro-organs, which are also referred to herein as “biopumps™”, may be implanted in animal model systems, and parameters and effects influenced by expression of the recombinant gene can be evaluated. In vitro expression can be assessed prior to implantation as well, enabling the possibility for in vitro to in vivo correlation studies of expressed recombinant proteins. Implantation of biopumps containing polynucleotides encoding at least two recombinant gene products, wherein one recombinant gene products differs by at least one amino acid from another recombinant gene product functioning as a protein-drug; provides an efficient and superior method for protein-drug optimization. Co-implantation of biopumps containing polynucleotides encoding at least two recombinant gene products, wherein the expression of one potentially functionally modifies or regulates the expression and/or function of the other, provides a completely novel method of determining in vivo modification and/or regulation effects between expressed recombinant products. These methods therefore provide for superior opportunities to assess recombinant gene product expression in vivo, in whole animal models, than what is currently available in the art.

While reducing the present invention to practice, it was found that in vivo expression of recombinant gene products could be accomplished utilizing genetically modified micro-organs or micro-organs. These micro-organs were configured of such dimensions as to enable their long-term upkeep in culture, and were found to remain structurally intact, and secrete high levels of recombinant proteins in vivo, following subsequent implantation within a host. This newly discovered method of protein and protein-drug expression is applicable for an infinite number of recombinant proteins in a variety of micro-organs, resulting in numerous almost unlimited applications evident from this novel technology, as further detailed hereunder.

It is one object of the present invention to provide a method of rapid assessment and validation of candidate protein-based therapeutic molecules encoded by nucleic acid sequences of interest.

It is another object of the present invention to provide a method of determining at least one pharmacological, physiological and/or therapeutic, quantitative or qualitative parameter or effect of an expressed recombinant gene product in vitro or in vivo.

It is yet another object of the present invention to provide a method of determining at least one pharmacological, physiological and/or therapeutic, quantitative or qualitative parameters or effects in an in vivo system utilizing micro-organs as a means of expressing nucleic acids of interest.

It is yet another object of the present invention to provide a method for assaying in vitro output levels of expressed recombinant gene products.

It is yet another object of the present invention to provide a method for assaying in vitro output levels of expressed recombinant gene products, and correlating them with in vivo expression levels to achieve an in vitro-in vivo correlation model.

It is yet another object of the present invention to provide a method of optimizing a protein-drug, wherein pharmacologic, physiologic and/or therapeutic, parameters or effects can be compared quantitatively or qualitatively, in vivo, for recombinant gene products differing by at least one amino acid from a protein-drug.

It is yet another object of the present invention to provide a method for determining pharmacologic, physiologic and/or therapeutic, parameters or effects quantitatively or qualitatively, for regulated recombinant gene products in vivo.

Thus, according to one aspect of the present invention there is provided a method of determining at least one quantitative or qualitative pharmacological, physiological and/or therapeutic, parameter or effect of a recombinant gene product in vivo, the method comprising (a) obtaining at least one micro-organ explant from a donor subject, the micro-organ explant comprising a population of cells, the micro-organ explant maintaining a microarchitecture of an organ from which it is derived and at the same time having dimensions selected so as to allow diffusion of adequate nutrients and gases to cells in the micro-organ explant and diffusion of cellular waste out of the micro-organ explant so as to minimize cellular toxicity and concomitant death due to insufficient nutrition and accumulation of the waste in the micro-organ explant, at least some cells of the population of cells of the micro-organ explant expressing and secreting at least one recombinant gene product; (b) implanting the at least one micro-organ explant in a recipient subject; and (c) determining the at least one quantitative or qualitative pharmacological, physiological and/or therapeutic, parameter or effect of the recombinant gene product in the recipient subject.

According to another aspect of the present invention there is provided a method of optimizing a protein-drug comprising (a) providing a plurality of polynucleotides encoding recombinant gene products differing by at least one amino acid from the protein-drug; (b) obtaining a plurality of micro-organ explants from a donor subject, each of the plurality of micro-organ explants comprises a population of cells, each of the plurality of micro-organ explants maintaining a microarchitecture of an organ from which it is derived and at the same time having dimensions selected so as to allow diffusion of adequate nutrients and gases to cells in the micro-organ explants and diffusion of cellular waste out of the micro-organ explants so as to minimize cellular toxicity and concomitant death due to insufficient nutrition and accumulation of the waste in the micro-organ explants; (c) genetically modifying the plurality of micro-organ explants, so as to obtain a plurality of genetically modified micro-organ explants expressing and secreting the proteins differing by the at least one amino acid; (d) implanting the plurality of genetically modified micro-organ explants within a plurality of recipient subjects; and (e) comparatively determining at least one pharmacological, physiological and/or therapeutic, quantitative or qualitative, parameters or effects of the proteins differing by the at least one amino acid in the recipient subject.

According to yet another aspect of the present invention there is provided a method of determining functional relations between recombinant gene products in vivo, the method comprising (a) providing at least one first polynucleotide encoding a first recombinant gene product; (b) providing at least one second polynucleotide encoding a second recombinant gene product whose expression potentially functionally modifies or regulates the expression and/or function of the first recombinant gene product; (c) obtaining a plurality of micro-organ explants from a donor subject, each of the plurality of micro-organ explants comprising a population of cells, each of the plurality of micro-organ explants maintaining a microarchitecture of an organ from which it is derived and at the same time having dimensions selected so as to allow diffusion of adequate nutrients and gases to cells in the micro-organ explants and diffusion of cellular waste out of the micro-organ explants so as to minimize cellular toxicity and concomitant death due to insufficient nutrition and accumulation of the waste in the micro-organ explants; (d) genetically modifying the plurality of micro-organ explants, so as to obtain a plurality of genetically modified micro-organ explants expressing and secreting the first and/or second recombinant gene products; (e) implanting the plurality of genetically modified micro-organ explants within a plurality of recipient subjects; and (f) determining the functional relations between the first and second recombinant gene products in vivo.

According to further features in the described preferred embodiments recombinant gene products may be of a known or unknown function.

According to still further features in the described preferred embodiments recombinant gene products may be of suspected function.

According to still further features in the described preferred embodiments recombinant gene products may be of suspected function based on sequence similarity to a protein of a known function.

According to further features in the described preferred embodiments recombinant gene products may be encoded by an expressed sequence tag (EST).

According to further features in the described preferred embodiments recombinant gene products may be encoded by a polynucleotide having a modified nucleotide sequence, as compared to a corresponding natural polynucleotide.

According to further features in the described preferred embodiments, some cells of the micro-organ explant express and secrete at least one recombinant gene product, as a result of genetic modification of at least a portion of the population of cells, by transfection with a recombinant virus carrying a recombinant gene encoding the recombinant gene product.

According to still further features in the described preferred embodiments, recombinant viruses carrying a recombinant gene encoding a recombinant gene product utilized for transfection of a population of cells of the explant may be selected from the group consisting of recombinant hepatitis virus, recombinant adenovirus, recombinant adeno-associated virus, recombinant papilloma virus, recombinant retrovirus, recombinant cytomegalovirus, recombinant simian virus, recombinant lenti virus and recombinant herpes simplex virus.

According to still further features in the described preferred embodiments genetic modification of at least some cells of the micro-organ explants to express and secrete at least one recombinant gene product can be accomplished by uptake of a non-viral vector carrying a recombinant gene encoding the recombinant gene product.

According to still further features in the described preferred embodiments, genetic modification of at least a population of cells of the micro-organ explant may be accomplished by cellular transduction with a foreign nucleic acid sequence via a transduction method selected from the group consisting of calcium-phosphate mediated transfection, DEAE-dextran mediated transfection, electroporation, liposome-mediated transfection, direct injection, gene gun transduction, pressure enhanced uptake of DNA and receptor-mediated uptake.

According to still further features in the described preferred embodiments, the recombinant gene product may be under the control of an inducible or constitutive promoter.

According to still further features in the described preferred embodiments, the recombinant gene product may be selected from the group consisting of recombinant proteins and recombinant functional RNA molecules.

According to still further features in the described preferred embodiments, recombinant gene products may, or may not be, normally produced by the organ from which the micro-organ explant is derived.

According to still further features in the described preferred embodiments, recombinant gene products may be encoded with a known tag peptide sequence to be introduced into the recombinant protein.

According to still further features in the described preferred embodiments, recombinant gene products may be encoded with a polycistronic recombinant nucleic acid including an IRES site sequence, a sequence encoding a reporter protein, and a sequence encoding the protein of interest.

According to still further features in the described preferred embodiments, recombinant proteins may be marker proteins.

According to still further features in the described preferred embodiments, recombinant proteins may be selected from the group consisting of natural or non-natural insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, ornithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the α family, transforming growth factors of the β family, gastrins, secretins, cholecystokinins, somatostatins, substance P and transcription factors.

According to still further features in the described preferred embodiments, micro-organ explants may be immune-protected by a biocompatible immuno-protective sheath.

According to still further features in the described preferred embodiments, implanting genetically modified micro-organs may be within an animal that is an established animal model for a human disease.

According to still further features in the described preferred embodiments, prior to biopump implantation in vivo, an in vitro secretion level of the gene product may be determined, and hence an in vitro-in vivo correlation model may be constructed to obtain a predetermined expression level in a given animal model.

According to still further features in the described preferred embodiments, the method of determining parameters or effects of recombinant gene products expressed in vivo by implanted micro-organ explants may be used for determining an in vivo effect of a protein-based drug.

According to still further features in the described preferred embodiments, pharmacokinetic, pharmacodynamic, physiologic and/or therapeutic parameters or effects of expressed recombinant proteins and/or protein-drug measured may include measurements in terms of efficacy, toxicity, mutagenicity, carcinogenicity and teratogenicity in vivo.

According to still further features in the described preferred embodiments, pharmacokinetic, pharmacodynamic, physiologic and/or therapeutic parameters or effects of expressed recombinant proteins and/or protein-drugs may be measured comparatively, and may include measurements in terms of efficacy, toxicity, mutagenicity, carcinogenicity and teratogenicity in vivo.

According to still further features in the described preferred embodiments determining functional relations between recombinant gene products comprises pharmacokinetic, pharmacodynamic, physiologic and/or therapeutic parameters or effects of expressed recombinant proteins and/or protein-drugs and may include measurements in terms of efficacy, toxicity, mutagenicity, carcinogenicity and teratogenicity in vivo.

According to still further features in the described preferred embodiments, determining at least one pharmacological, physiological and/or therapeutic, quantitative or qualitative, parameters or effects of the recombinant gene product in the animal include determining animal survival and/or animal pathogen burden.

According to still further features in the described preferred embodiments, determining at least one pharmacological, physiological and/or therapeutic, quantitative or qualitative, parameters or effects of the recombinant gene product in terms of protein functional relations in the animal include determining animal survival and/or animal pathogen burden.

According to still further features in the described preferred embodiments, determining at least one pharmacological, physiological and/or therapeutic, quantitative or qualitative, parameters or effects of the recombinant gene product comparatively in the animal include determining relative animal survival and/or animal pathogen burden.

According to still further features in the described preferred embodiments, comparatively determining quantitative or qualitative pharmacological, physiological and/or therapeutic parameters or effects recombinant gene products in recipient subjects comprises protein-drug synergistic effects.

According to still further features in the described preferred embodiments, comparatively determining quantitative or qualitative pharmacological, physiological and/or therapeutic parameters or effects recombinant gene products in recipient subjects comprises protein-drug antagonistic effects

According to still further features in the described preferred embodiments, determining functional relations between recombinant gene products comprises determining the level of RNA expression of one recombinant gene product in the presence and absence of another recombinant gene product.

According to still further features in the described preferred embodiments, determining functional relations between recombinant gene products comprises determining a level of protein expression of one recombinant gene product in the presence and absence of another recombinant gene product.

According to still further features in the described preferred embodiments, determining functional relations between recombinant gene products comprises determining a level of activity of one recombinant gene product in the presence and absence of another recombinant gene product.

According to still further features in the described preferred embodiments determining functional relations between recombinant gene products comprises determining direct effects of one recombinant gene product on another. Such direct effects may comprise functional and/or structural modification of a recombinant gene product, including cleavage, phosphorylation, glycosylation, methylation or assembly of a recombinant gene product. Functional and/or structural modification may also comprise the processing of a recombinant gene product to its active form.

According to still further features in the described preferred embodiments determining functional relations between recombinant gene products comprises determining indirect effects of one recombinant gene product on another. Such indirect effects may comprise functional and/or structural modification of a recombinant gene product, including positive or negative effects on promoter sequences, and these effects may be mediated in trans.

According to still further features in the described preferred embodiments, the dimensions of the explant are selected as such that cells positioned deepest within said micro-organ explant are at least about 125-150 micrometers and not more than about 225-250 micrometers away from the nearest surface of the micro-organ explant.

According to still further features in the described preferred embodiments, the dimensions of the explant are selected as such that the explant has a surface area to volume index characterized by the formula 1/x+1/a>1.5 mm-1; wherein ‘x’ corresponds to tissue thickness and ‘a’ corresponds to the width of the tissue in millimeters.

According to still further features in the described preferred embodiments, the organ is selected from the group consisting of lymph organ, pancreas, liver, gallbladder, kidney, digestive tract organ, respiratory tract organ, reproductive organ, skin, urinary tract organ, blood-associated organ, thymus or spleen.

According to still further features in the described preferred embodiments, genetically modified micro-organ explants comprising epithelial and connective tissue cells are arranged in a microarchitecture similar to the microarchitecture of the organ from which the explant is obtained.

According to still further features in the described preferred embodiments, genetically modified micro-organ explants derived from the pancreas may include modification of a population of islet of Langerhan cells.

According to still further features in the described preferred embodiments, genetically modified micro-organ explants derived from the skin may include at least one hair follicle and gland.

According to still further features in the described preferred embodiments, genetically modified micro-organ explants may be derived from diseased skin, and the explant may include a population of hyperproliferative or neoproliferative cells from the diseased skin.

According to still further features in the described preferred embodiments, genetically modified micro-organ explants may be derived from a donor subject, or the recipient.

According to still further features in the described preferred embodiments, genetically modified micro-organ explants may be derived from a human being, or from a non-human animal.

According to still further features in the described preferred embodiments, the recipient of the genetically modified micro-organ may be a human being, or a non-human animal.

According to still further features in the described preferred embodiments, at least some cells of the population of cells of the micro-organ explants express and secrete at least one recombinant gene product in a continuous, sustained fashion.

According to still further features in the described preferred embodiments, at least some cells of the population of cells of the micro-organ explants express and secrete at least one recombinant gene product in a continuous, sustained fashion, following administration of an inducing agent.

According to still further features in the described preferred embodiments, at least some cells of the population of cells of the micro-organ explants cease to express and secrete the recombinant gene product, following removal of the inducing agent.

According to still further features in the described preferred embodiments, at least some cells of said population of cells of said micro-organ explant cease to express and secrete said at least one recombinant gene product, following administration of a repressor agent.

According to still further features in the described preferred embodiments determining quantitative or qualitative pharmacological, physiological and/or therapeutic, parameters or effects of recombinant gene products in a recipient subject comprises using at least one of the following assays: ELISA, Western blot analysis, HPLC, mass spectroscopy, GLC, immunohistochemistry, RIA, metabolic studies, patch-clamp analysis, perfusion assays, PCR, RT-PCR, Northern blot analysis, Southern blot analysis, RFLP analysis, nuclear run-on assays, gene mapping, cell proliferation assays and cell death assays.

Thus the present invention successfully addresses the shortcomings of the presently known configurations by providing a method of genetically modifying cells within a micro-organ explant to express recombinant gene products, which can be used to measure in vitro production, or can be implanted within a host in order to analyze in vivo production of the recombinant gene product. Combinatorial effects, as well as functional and regulation effects can be uniquely assessed using this unprecedented system.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graphic representation revealing high levels of mEPO transgene incorporation in human skin micro-organs (MOs) transfected with pORF-hEPO-plasmids. By 4 days post-transfection MO maintenance of the transgene is high, however by 18 days post-transfection transgene expression is not detected. Inactivation of endogenous DNases minimally prolongs transgene expression, with 1 ng/ml the concentration with best results. Centrifugation did not enhance, and may even have hindered efficient transgene incorporation.

FIG. 2 is a graphic representation revealing high levels of in vitro secretion of mouse erythropoietin (mEPO) from human skin micro-organs (MOs) transduced with mEPO, that are dose-dependant, as compared to controls. In vitro production occurred as late as 88 days.

FIG. 3A is a graphic representation revealing high circulating mIFNα levels in serum of mice implanted with human skin biopumps expressing the mIFNα gene, as compared to control mice implanted with biopumps expressing the lacZ reporter gene (serum collected on days 4, 14, 24 and 35 post implantation).

FIG. 3B is a graphic representation of a correlation between data representing in vitro production of mIFNα as a function of the number of nanograms of protein produced per unit time, per micro-organ cultured (ng/day/MO) and data representing in vivo production of mIFNα as a function of the number of picograms of protein detected per ml of blood collected following implantation. In vivo mIFNα production data correlated directly with in vitro MO production.

FIG. 4 is a graphic representation plotting secreted mIFNα levels assayed from serum of mice implanted with mIFNα expressing MOs versus data is collected by a viral cytopathic inhibition assay. Inhibition of viral cytopathic effects was measured according to correspondence of serum activity levels, with that of values generated by a standard curve of parallel administration of purified recombinant mIFNα to infected LKT cells. Viral cytopathic activity almost directly paralleled that of mIFNα circulating levels, indicating a causal relationship

FIG. 5A is a micrograph revealing intact structural integrity of mouse lung biopumps (arrow) implanted subcutaneously in C57B1/6 mice, 140 days post implantation.

FIG. 5B is a micrograph revealing intact structural integrity of another mouse lung biopump (arrow) implanted subcutaneously in C57B1/6 mice, 140 days post implantation.

FIG. 5C is a micrograph revealing intact structural integrity of an additional mouse lung biopump following implantation in C57B1/6 mice, 174 days post implantation.

FIG. 6 is a micrograph revealing intact structural integrity of human skin biopumps (arrow) 76 days following their implantation subcutaneously in SCID mice.

### DESCRIPTION OF THE PREFERRED EMBODIMENTS

The present invention provides a novel and superior method of assessing and validating candidate protein-based therapeutic molecules. The method utilizes genetically modified micro-organs, also referred to herein as biopumps™, to express nucleic acid sequences of interest, encoding putative nucleic acid or protein-drugs. The use of genetically modified micro-organs provides a means of efficient determination of pharmacological, physiological and/or therapeutic parameters or effects of the candidate molecule in vitro and/or in vivo.

Genetically modified micro-organs, or biopumps, may be implanted in animal model systems, and effects and parameters influenced by expression of the recombinant gene can be evaluated.

Moreover, the methods disclosed herein provide a means to assess multiple candidates simultaneously, and enable assessment of cross-regulation effects, synergistic or antagonistic effects among candidate drugs.

These effects can be assessed quantitatively or qualitatively. In vitro expression can be assessed prior to implantation as well, enabling the possibility for in vitro to in vivo correlation studies of expressed recombinant proteins.

These methods therefore provide for superior opportunities to assess recombinant gene product expression in vivo, in whole animal models, than what is currently available in the art.

The principles and operation of the methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In one preferred embodiment of the present invention a method is disclosed for obtaining micro-organs from a donor individual, genetically modifying the micro-organs to express a recombinant product, delivering the genetically modified micro-organs to a recipient subject, and measuring a qualitative or quantitative, physiologic, pharmacologic or therapeutic parameter or effect of the recombinant product within the recipient subject.

### Obtaining Micro-Organs from a Donor Individual

Methods for the preparation and processing of micro-organs into genetically modified “biopumps” are disclosed in PCT/IL01/00976, EP Application No. 01204125.7 and U.S. patent application Ser. Nos. 08/783,903 and 09/589,736, which are incorporated herein by reference, to comprise tissue dimensions defined such that diffusion of nutrients and gases into every cell in the three dimensional micro-organ, and sufficient diffusion of cellular wastes out of the explant, is assured. Ex-vivo maintenance of the micro-organs in minimal media can continue for an extended period of time, whereupon controlled ex-vivo transduction incorporating desired gene candidates within cells of the micro-organs using viral or non-viral vectors occurs, thus creating genetically modified micro-organs or “biopumps”.

This novel and versatile technology may be used for qualitative or quantitative assaying of in vitro expression and/or secretion levels of the desired protein from the biopumps.

In a preferred embodiment of the present invention, in vitro-to-in vivo correlation models can be developed once the in vitro output expression and/or secretion levels of the desired protein from the biopumps has been determined; whereby in vivo serum levels and/or physiological responses can be estimated based on their in vitro expression and/or secretion levels. Regulation of downstream effects as a result of the treatment can be evaluated, as well.

As used herein, the term “micro-organ” refers to organ tissue which is removed from a body and which is prepared, as is further described below, in a manner conducive for cell viability and function. Such preparation may include culturing outside the body for a predetermined time period. Micro-organs retain the basic micro-architecture of the tissues of origin. The isolated cells together form a three dimensional structure which simulates/retains the spatial interactions, e.g., cell-cell, cell-matrix and cell-stromal interactions, and the orientation of actual tissues and the intact organism from which the explant was derived. Accordingly, such interactions as between stromal and epithelial layers is preserved in the explanted tissue such that critical cell interactions provide, for example, autocrine and paracrine factors and other extracellular stimuli which maintain the biological function of the explant. Concurrently, micro-organs are prepared such that cells positioned deepest within a micro-organ are at least about 125-150 micrometers and not more than about 225-250 micrometers away from a nearest source of nutrients, gases, and waste sink, thereby providing for the ability to function autonomously and for long term viability both as ex-vivo cultures and in the implanted state. Micro-organ dimensions can be calculated to comprise a surface area to volume index characterized by the formula 1/x+1/a>1.5 mm-1; wherein ‘x’ represents the tissue thickness and ‘a’ represents the tissue width, in millimeters. These dimensions, as above, enable the efficient diffusion of nutrients and gases to the cells of the micro-organ, and concurrently allow for efficient waste removal.

### Source of Explants for the Micro-Organ

Examples of donor mammals from which the micro-organs can be isolated include humans and other primates, swine, such as wholly or partially inbred swine (e.g., miniature swine, and transgenic swine), rodents, etc. Micro-organs may be processed from tissue from a variety of organs, including: the lymph system, the pancreas, the liver, the gallbladder, the kidney, the pancreas, the digestive tract, the respiratory tract, the reproductive system, the skin, the urinary tract, the blood, the bladder, the cornea, the prostate, the bone marrow, the thymus and the spleen. Explants from these organs can comprise, but are not excluded to, islet of Langerhan cells, hair follicles, glands, epithelial and connective tissue cells, arranged in a microarchitecture similar to the microarchitecture of the organ from which the explant was obtained.

For convenience, certain terms employed in the specification, examples, and appended claims are collected here.

The term “tissue” refers to a group or layer of similarly specialized cells, which together perform certain special functions.

The term “organ” refers to two or more adjacent layers of tissue, which layers of tissue maintain some form of cell-cell and/or cell-matrix interaction to generate a microarchitecture. In the present invention, micro-organ cultures were prepared from such organs as, for example, mammalian skin, mammalian pancreas, liver, kidney, duodenum, esophagus, thymus and spleen.

The term “stroma” refers to the supporting tissue or matrix of an organ.

The term “isolated” as used herein refers to an explant, which has been separated from its natural environment in an organism. This term includes gross physical separation from its natural environment, e.g., removal from the donor animals, e.g., a mammal such as a human or a miniature swine. For example, the term “isolated” refers to a population of cells, which is an explant, is cultured as part of an explant, or is transplanted in the form of an explant. When used to refer to a population of cells, the term “isolated” includes population of cells, which results from proliferation of cells in the micro-organ culture of the invention.

The terms “epithelia” and “epithelium” refer to the cellular covering of internal and external body surfaces (cutaneous, mucous and serous), including the glands and other structures derived therefrom, e.g., corneal, esophageal, epidermal and hair follicle epithelial cells. Other exemplary epithelial tissues include: olfactory epithelium, which is the pseudostratified epithelium lining the olfactory region of the nasal cavity, and containing the receptors for the sense of smell; glandular epithelium, which refers to epithelium composed of secreting cells; squamous epithelium, which refers to epithelium composed of flattened plate-like cells. The term epithelium can also refer to transitional epithelium, which is that characteristically found lining hollow organs that are subject to great mechanical change due to contraction and distention, e.g., tissue that represents a transition between stratified squamous and columnar epithelium.

The term “skin” refers to the outer protective covering of the body, consisting of the dermis and the epidermis, and is understood to include sweat and sebaceous glands, as well as hair follicle structures.

The term “gland” refers to an aggregation of cells specialized to secrete or excrete materials not related to their ordinary metabolic needs. For example, “sebaceous glands” are holocrine glands in the corium that secrete an oily substance and sebum.

The term “sweat glands” refers to glands that secrete sweat, situated in the corium or subcutaneous tissue, opening by a duct on the body surface. The ordinary or eccrine sweat glands are distributed over most of the body surface, and promote cooling by evaporation of the secretion; the apocrine sweat glands empty into the upper portion of a hair follicle instead of directly onto the skin, and are found only in certain body areas, as around the anus and in the axilla.

The term “hair” (or “pilus”) refers to a threadlike structure; especially the specialized epidermal structure composed of keratin and developing from a papilla sunk in the corium, produced only by mammals and characteristic of that group of animals. A “hair follicle” refers to one of the tubular-invaginations of the epidermis enclosing the hairs, and from which the hairs grow; and “hair follicle epithelial cells” refers to epithelial cells which are surrounded by the dermis in the hair follicle, e.g., stem cells, outer root sheath cells, matrix cells, and inner root sheath cells. Such cells may be normal non-malignant cells, or transformed/immortalized cells.

An additional source for micro-organ explants may also be from diseased tissue, whereby the explant comprises a population of hyperproliferative, neoproliferative or transformed cells. As transduction of the cells of the micro-organ for production of a recombinant gene product is essential, hyperproliferating or neoproliferating cells provide additional benefits for transduction, including a greater possibility for incorporation of retroviral vectors, as well as a potential for greater recombinant product output, as will be discussed hereinbelow.

Accordingly, as used herein, “proliferative”, “proliferating” and “proliferation” refer to cells undergoing mitosis.

As used herein, “transformed cells” refers to cells, which have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control.

As used herein the term “donor” refers to a subject, which provides the cells, tissues, or organs, which are to be placed in culture and/or transplanted to a recipient subject. Donor subjects can also provide cells, tissues, or organs for reintroduction into themselves, i.e., for autologous transplantation.

In one preferred embodiment of this invention, donor subjects for the generation of micro-organs include humans, non-human primates, swine, such as wholly or partially inbred swine (e.g., miniature swine, and transgenic swine), rodents, sheep, dogs, cows, chickens, amphibians, reptiles, and other mammals.

### Genetically Modifying the Micro-Organs to Express a Recombinant Product

Incorporation of recombinant nucleic acid within the micro-organs to generate genetically modified micro-organs or biopumps can be accomplished through a number of methods well known in the art. Nucleic acid constructs can be utilized to stably or transiently transduce the micro-organ cells. In stable transduction, the nucleic acid molecule is integrated into the micro-organ cells genome and as such it represents a stable and inherited trait. In transient transduction, the nucleic acid molecule is maintained in the transduced cells as an episome and is expressed by the cells but it is not integrated into the genome. Such an episome can lead to transient expression when the transduced cells are rapidly dividing cells due to loss of the episome or to long term expression wherein the transduced cells are non-dividing cells such as for example muscle cells transduced with Adeno vector gave an expression of the transgene for more than a year.

Typically the nucleic acid sequence is subcloned within a particular vector, depending upon the preferred method of introduction of the sequence to within the micro-organs. Once the desired nucleic acid segment is subcloned into a particular vector it thereby becomes a recombinant vector. To generate the nucleic acid constructs in context of the present invention, the polynucleotide segments encoding sequences of interest can be ligated into commercially available expression vector systems suitable for transducing mammalian cells and for directing the expression of recombinant products within the transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

According to a preferred embodiment of the present invention, recombinant products are introduced by genetic modification of a population of cells of one or more of the micro-organ explants accomplished by cellular transduction with a foreign nucleic acid sequence.

There are a number of techniques known in the art for introducing the above described recombinant vectors into the cells of structures such as the micro-organs used in the present invention, such as, but not limited to: direct DNA uptake techniques, and virus, plasmid, linear DNA or liposome mediated transduction, receptor-mediated uptake and magnetoporation methods employing calcium-phosphate mediated and DEAE-dextran mediated methods of introduction, electroporation, liposome-mediated transfection, direct injection, and receptor-mediated uptake (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals). Micro-organ bombardment with nucleic acid coated particles is also envisaged.

In another preferred embodiment of the present invention, exogenous polynucleotide introduction into micro-organs is via ex-vivo transduction of the cells with a viral or non-viral vector encoding the sequence of interest.

### Ex-Vivo Viral Transduction (Transfection)

Incorporation of desired gene candidates into the cells of the micro-organs to create genetically modified micro-organd, or biopumps, can be accomplished using various viral vectors. The viral vector is engineered to contain nucleic acid, e.g., a cDNA, encoding the desired gene product. Transfection of cells with a viral vector has the advantage that a large proportion of cells receive the nucleic acid which can obviate the need for selection of cells which have received the nucleic acid. Additionally, molecules encoded within the viral vector, e.g., a cDNA contained in the viral vector, are expressed efficiently in cells which have taken up viral vector nucleic acid and viral vector systems can be used either in vitro or in vivo.

Defective retroviruses are well characterized for use in gene transfer for gene therapy purposes (for review see Miller, A. D. (1990) Blood 76:271). A recombinant retrovirus can be constructed having a nucleic acid encoding a gene product of interest inserted into the retroviral genome. Additionally, portions of the retroviral genome can be removed to render the retrovirus replication defective. The replication defective retrovirus is then packaged into virions which can be used to infect a target cell through the use of a helper virus by standard techniques.

Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danosand Mulligan (1988) Proc. Natl. Acad. Sci. USA 85:6460-6464; Wilson et al. (1988) Proc. Natl. Acad. Sci USA 85:3014-3018; Armentano et al., (1990) Proc. Natl. Acad. Sci. USA 87: 6141-6145; Huber et al. (1991) Proc. Natl. Acad. Sci. USA 88:8039-8043; Feri et al. (1991) Proc. Natl. Acad. Sci. USA 88:8377-8381; Chowdhury et al. (1991) Science 254:1802-1805; van Beusechem et al. (1992) Proc. Natl. Acad. Sci USA 89:7640-7644; Kay et al. (1992) Human Gene Therapy 3:641-647; Dai et al. (1992) Proc. Natl. Acad. Sci. USA 89:10892-10895; Hwu et al (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cells of the micro-organs.

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and to foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol 57:267). Most replication-defective adenoviral vectors currently in use are deleted for all or parts of the viral E1 and E3 genes but retain as much as 80% of the adenoviral genetic material.

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics In Micro. And Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introdue DNA into cells. A variety of nucleic acids have been introduced into different cell types using AAV vectors (see for example Hermonat et al. (1984)Proc. Natl. Acad. Sci. USA 81:6466-6470; Tratschin et al. (1985) Mol. Cell Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

Therefore, according to a preferred embodiment of the present invention, and not by way of limitation, the vector employed can be Adeno-associated virus (AAV) [For a review see Muzyczka et al. Curr. Topics In Micro. And Immunol. (1992) 158:97-129; Flotte et al. (1992) Am. J. Respir. Cell. Mol. Biol. 7:349-356; Samulski et al. (1989) J. Virol. 63:3822-3828; and McLaughlin et al (1989) J. Virol. 62:1963-1973; Tratschin et al. (1985) Mol. Cell. Biol. 5:3251-3260)]; Murine Leukemia Virus (MuLV) [See for example, Wang G. et al Curr Opin Mol Ther 2000 October;2-5:497-506; Guoshun Wang et al, ASGT 2001 Abst.]; Adenovirus [See for example Berkner et al. (1988) BioTechniques 6:616; Rosenfeld et al. (1991) Science 252:431-434; and Rosenfeld et al. (1992) Cell 68:143-155.] Suitable adenoviral vectors derived from the adenovirus strain, such as Ad type 5 dl324 or other strains of adenovirus (e.g., Ad2, Ad3, Ad7 etc.) are well known to those skilled in the art; and Lenti virus [see for example, Wang G. et al Curr Opin Mol Ther 2000 October;2-5:497-506], and are additional preferred embodiments of the present invention, as are viral vectors comprising recombinant hepatitis virus, recombinant papilloma virus, recombinant retrovirus, recombinant cytomegalovirus, recombinant simian virus, recombinant lenti virus and recombinant herpes simplex virus.

When using a viral vector, the nucleic acid segment encoding the protein in question and the necessary regulatory elements have been incorporated into the viral genome (or partial viral genome).

### Ex-Vivo Transduction with Non-Viral Vectors (Transformation)

Non-viral vectors may also be used to transduce the cells of the micro-organs with recombinant nucleic acids to yield genetically modified micro-organs, or biopumps, and are additional preferred embodiments of the present invention. These sequences may also be engineered to include the necessary regulatory elements within the non-viral vector. Examples of such non-viral vectors include, and not by way of limitation: Plasmids such as CDM8 (Seed, B. (1987) Nature 329:840) and pMT2PC (Kaufman, et al. (1987) EMBO J. 6:187-195). Additional suitable commercially available mammalian expression vectors include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives. Linear DNA expression cassettes (LDNA) may be employed as well (Zhi-Ying Chen et al ASGT 2001 Abst.)

Nucleotide sequences which regulate expression of a gene product (e.g., promoter and enhancer sequences) are selected based upon the type of cell in which the gene product is to be expressed and the desired level of expression of the gene product. For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements which are known in the art include upstream regions from the dystrophin gene (Klamut et al., (1989) Mol. Cell Biol.9:2396), the creatine kinase gene (Buskin and Hauschka, (1989) Mol. Cell Biol. 9:2627) and the troponin gene (Mar and Ordahl, (1988) Proc. Natl. Acad. Sci. USA. 85:6404).

Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters). Alternatively, a regulatory element which can direct constitutive expression of a gene in a variety of different cell types, such as a viral regulatory element, can be used. Examples of viral promoters commonly used to drive gene expression include those derived from polyoma virus, Adenovirus 2, cytomegalovirus and Simian Virus 40, and retroviral LTRs.

Alternatively, a regulatory element which provides inducible expression of a gene linked thereto can be used. The use of an inducible regulatory element (e.g., an inducible promoeter) allows for modulation of the production of the gene product in the cell. Examples of potentially useful inducible regulatory systems for use in eukaryotic cells include hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA89:1014-10153). Additional tissue-specific or inducible regulatory systems which may be developed can also be used in accordance with the invention.

Therefore according to further features of a preferred embodiment of the present invention, the recombinant gene product may be under the control of an inducible or constitutive promoter.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product which is easily detectable and, thus, can be used to evaluate efficacy of the system. Standard reporter genes used in the art include genes encoding β-galactosidase, chloramphenicol acetyl transferase, luciferase and human growth hormone.

In another preferred embodiment of this invention, polynucleotide(s) can also include trans-, or cis-acting enhancer or suppresser elements which regulate either the transcription or translation of endogenous genes expressed within the cells of the micro-organs, or additional recombinant genes introduced into the micro-organs. Numerous examples of suitable translational or transcriptional regulatory elements, which can be utilized in mammalian cells, are known in the art.

For example, transcriptional regulatory elements comprise cis- or trans-acting elements, which are necessary for activation of transcription from specific promoters [(Carey et al., (1989), J. Mol. Biol. 209:423-432; Cress et al., (1991), Science 251:87-90; and Sadowski et al., (1988), Nature 335:5631-564)].

Translational activators are exemplified by the cauliflower mosaic virus translational activator (TAV) [see for example, Futterer and Hohn, (1991), EMBO J. 10:3887-3896]. In this system a bi-cistronic mRNA is produced. That is, two coding regions are transcribed in the same mRNA from the same promoter. In the absence of TAV, only the first cistron is translated by the ribosomes, however, in cells expressing TAV, both cistrons are translated.

The polynucleotide sequence of cis-acting regulatory elements can be introduced into cells of micro-organs via commonly practiced gene knock-in techniques. For a review of gene knock-in/out methodology see, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO93/14200, WO 94/06908 and WO 94/28123 also provide information.

Down-regulation of endogenous sequences may also be desired, in order to assess production of the recombinant product exclusively. Toward this end, antisense RNA may be employed as a means of endogenous sequence inactivation. Exogenous polynucleotide(s) encoding sequences complementary to the endogenous mRNA sequences are transcribed within the cells of the micro-organ. Down regulation can also be effected via gene knock-out techniques, practices well known in the art (“Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988)).

Overexpression of the recombinant product may be desired as well. Overexpression may be accomplished by providing a high copy number of one or more coding sequences in the respective vectors. These exogenous polynucleotide sequences can be placed under transcriptional control of a suitable promoter of a mammalian expression vectors to regulate their expression.

### Recombinant Product Expression

Recombinant product expression can provide for functional RNA molecule or protein production, and is a preferred embodiment of the present invention. Biopump expression of the recombinant product can be verified in vitro, at the level of gene expression, by methods widely known in the art, including, but not limited to Northern blot analysis, RT-PCR assays and RNA protection assays, and other hybridization techniques.

In vitro protein production can be verified by methods including, but not limited to, HPLC, mass spectroscopy, GLC, immunohistochemistry, ELISA, RIA, or western blot analysis. When using a method which relies on the immunological properties of the protein in question, polyclonal antibodies against the entire protein or a peptide derived from can be raised and used. Alternatively, and according to a preferred embodiment of the present invention, an expressed sequence tag (EST) encoding a known tag peptide sequence (for example HIS tag) can be inserted into the recombinant protein either on the 5′ or the 3′ end thus the HIS-tag proteins can be isolated using His-Tag Ni-column chromatography. Similarly, in still another preferred embodiment of the present invention, a polycistronic recombinant nucleic acid including an IRES site sequence residing between the sequence encoding the protein of interest and a sequence encoding a reporter protein may be generated, so as to enable detection of a known marker protein. Additional marker proteins may be incorporated, or comprise the recombinant proteins, and as such encompass still further preferred embodiments of the present invention.

If the protein in question affects metabolic function then a typical method for analysis would be conducting metabolic studies, including recombinant product/protein-drug perfusion assays. If the protein in question affects cell membrane potential, then a typical method for analysis would be patch clamp analysis. If the protein in question is an enzyme with a known enzymatic activity, a typical method for analysis would be enzyme-substrate analysis. If the protein in question takes part in a ligand-receptor relationship, a ligand receptor analysis may be performed. Lastly, if the protein in question affects cell turnover, then a typical method for analysis would be conducting cell proliferation/differentiation assays. With any of the aforementioned methods, the result can either be quantitative (i.e., the numerical value obtained) or qualitative (e.g., detected or non-detected, implying a pre-set threshold of detection).

Another in-vivo function of the expressed recombinant products may be to affect gene expression. These effects may be analyzed by methods comprising PCR, RT-PCR, Northern blot analysis, Southern blot analysis, RFLP analysis, nuclear run-on assays, gene mapping, cell proliferation assays and cell death assays and encompass yet another preferred embodiment of the present invention.

All the above listed methods may be employed for in vivo verification of production and function of the recombinant protein or functional RNA molecule. RNA may be extracted from tissue and analyzed by the above methods, as well as by in situ hybridization techniques. Protein production may be analzed from organ homogneates, serum, plasma and lymph, via the methods outlined above.

Similarly, parameters involved in and/or effects of in vivo production of recombinant protein or functional RNA molecules produced by implanted biopumps may be measured via the methods disclosed hereinabove, and their measurement as such provide additional preferred embodiments of the present invention.

According to yet another preferred embodiment of the present invention, the recombinant protein-drug candidates may include an insulin, an amylase, a protease, a lipase, a kinase, a phosphatase, a glycosyl transferase, trypsinogen, chymotrypsinogen, a carboxypeptidase, a hormone, a ribonuclease, a deoxyribonuclease, a triacylglycerol lipase, phospholipase A2, elastase, amylase, a blood clotting factor, UDP glucuronyl transferase, ornithine transcarbamoylase, cytochrome p450 enzyme, adenosine deaminase, serum thymic factor, thymic humoral factor, thymopoietin, a growth hormone, a somatomedin, a costimulatory factor, an antibody, a colony stimulating factor, erythropoietin, epidermal growth factor, hepatic erythropoietic factor (hepatopoietin), a liver-cell growth factor, an interleukin, an interferon, a negative growth factor, a fibroblast growth factor, a transforming growth factor of the α family, a transforming growth factor of the β family, gastrin, secretin, cholecystokinin, somatostatin, serotinin, substance P, a signaling molecule, an intracellular trafficking molecule, a cell surface receptor, a cell surface receptor agonist, a cell surface receptor antagonist, a ribozyme and a transcription factor.

According to yet another preferred embodiment of the present invention, the recombinant protein-drug candidates may include recombinant gene products of a known or unknown function, of a suspected function or of suspected function based on sequence similarity to a protein of a known function.

The sequencing of a variety of organism genomes, including bacterial, yeast and the human genome has provided a wealth of information regarding, among other things, protein sequence information. Once the analysis of a completed, fully assembled genome occurs, it is possible to determine all the putative open reading frames (ORFs), which may constitute protein coding regions. These derived amino acid sequences are searched against sequence databases of other previously sequenced organisms, in order to determine the relationship to previously sequenced genes, in an attempt to correlate the proteins functions, based on these sequence homologies. There can be three results to these types of searches, a high degree of homology of the gene of interest with a previously sequenced gene encoding a protein of known function, a high degree of homology of the gene of interest with a previously sequenced gene encoding a protein of unknown function (usually referred to as a conserved hypothetical protein), or no database match. In cases of homology to genes encoding proteins of known function, the newly sequenced gene is generally annotated as a homologue of the “best fit” (Henikoff S, and Henikoff J G. (1994) Genomics 19: 97-107). Yet when the first bacterial genome sequences were elucidated, it was surprising that a significant percentage (35%-45%) of identified ORFs were either of unknown function or had no database match. More surprising is that these numbers have not changed substantially as more and more sequences have been determined (Weinstock, G. M. (2000) Emerging infectious disease 6(5): 496-505; Himmelreich R, Plagens H, Hilbert H, Reiner B, Herrmann R. (1997) Nucleic Acids Res 25: 701-12). Thus, close to half of all bacterial ORFs identified to date have no known function, half of which are unique to the given species. This represents an enormous storehouse of unrecognized metabolic potential, and it appears obvious that many novel biochemical reactions and pathways are yet to be discovered and characterized.

Whole genome studies may be applied to predicting the function of genes (Akerley B J, Rubin E J, Camilli A, Lampe D J, Robertson H M, Mekalanos J J. (1998) Proc Natl Acad Sci USA 95:8927-32). Predictions of gene function, a key step in the annotation of genomes, is essential for understanding particular gene and protein function in health and disease. Predictions are frequently made by assigning the uncharacterized gene the annotated function of the gene it is most similar to (similarity is measured by a database searching programs such as BLAST, DOMAIN, BEAUTY (BLAST Enhanced Alignment Utility), GENPEPT and TREMBL), or through information about the evolutionary relationships of the uncharacterized gene, according to their position in the tree relative to genes with known functions and according to evolutionary events (such as gene duplications) that may identify groups of genes with similar functions (Herrmann R, Reiner B. (1998) Curr Opin Microbiol 1:572-9).

According to still other preferred embodiments of the present invention, recombinant gene products may be of natural or non-natural proteins. Natural proteins may be selected from a variety of sources naturally produced in living systems, such as the examples listed hereinabove, and others. Non-natural proteins, however, as referred to herein, comprise proteins encoded by polynucleotide sequences that have been mutated, as compared to their natural counterpart. Numerous strategies to achieve production of a mutated, nonnatural protein are well known and practiced in the art, including chemical and insertional and site-directed mutagenesis.

Evolutionary protein design is a recently developed additional approach toward generating protein products, referred to herein as “evolved proteins” differing from their natural counterparts by alteration of the amino acid sequence and therefore their properties, through appropriate modifications at the DNA level. (Evolutionary Protein Design (2000) volume 55, Advances in Protein Chemistry, Academic press, ed. F. H. Arnold). Evolutionary protein design is a directed molecular evolutionary process, whereby the underlying process has a defined goal, and the key processes—mutation, recombination and screening or selection—are controlled by the experimenter.

Methods producing evolved proteins include modified methods for gene recombination events. DNA shuffling methods producing evolved proteins is achieved through random priming recombination (RPR) events (Z. Shao, H. Zhao, L. Giver and F. H. Arnold, (1998) Nucleic Acids Research, 26: 681-683, Crameri A., Raillard S. A., Bermudez E. and Stemmer W. P. C. (1998) Nature 391: 288-291), whereby short polynucleotide fragments are generated by primer extension along template strands. A staggered extension process (StEP) (H. Zhao, L. Giver, Z. Shao, J. A. Affholter and F. H. Arnold, (1998) Nature Biotechnology 16: 258-262) follows, whereby after denaturation, the primers re-anneal randomly to the templates and re-extend them, and heteroduplex recombination following repeat denaturation and extension results in the production of full length genes (A. Volkov, Z. Shao and F. H. Arnold, (1999) Nucleic Acids Research, 27: e18). These altered genes are cloned back into a plasmid for expression in a suitable host organism (bacteria or yeast). Clones expressing altered or evolved proteins are identified in a high-throughput screen, or in some cases, by selection, and the gene(s) encoding the evolved proteins are isolated and may in turn be recycled for additional rounds of directed evolution, as the need arises.

Thus, according to still other preferred embodiments of the present invention, recombinant gene products may be encoded by a polynucleotide having a modified nucleotide sequence, as compared to a corresponding natural polynucleotide.

In addition to proteins, recombinant gene products may also comprise functional RNA molecules.

### Functional RNA Molecules

According to another preferred embodiment of the present invention there is provided a method of generating functional RNA molecules within micro-organs. Functional RNA molecules can comprise antisense oligonucleotide sequences, ribozymes comprising the antisense oligonucleotide described herein and a ribozyme sequence fused thereto. Such a ribozyme is readily synthesizable using solid phase oligonucleotide synthesis.

Ribozymes are being increasingly used for the sequence-specific inhibition of gene expression by the cleavage of mRNAs encoding proteins of interest [Welch et al., “Expression of ribozymes in gene transfer systems to modulate target RNA levels.” Curr Opin Biotechnol. 1998 October;9(5):486-96]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., “Ribozyme gene therapy for hepatitis C virus infection.” Clin Diagn Virol. Jul. 15, 1998;10(2-3):163-71.]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms has demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

### Delivering the Genetically Modified Micro-Organ to a Recipient Animal

Micro-organ implantation within a recipient subject provides for a sustained dosage of the recombinant product. The micro-organs may be prepared, prior to implantation, for efficient incorporation within the host facilitating, for example, formation of blood vessels within the implanted tissue. Recombinant products may therefore be delivered immediately to peripheral recipient circulation, following production. Alternatively, micro-organs may be prepared, prior to implantation, to prevent cell adherence and efficient incorporation within the host. Examples of methods that prevent blood vessel formation include encasement of the micro-organs within commercially available cell-impermeant diameter restricted biological mesh bags made of silk or nylon, or others such as, for example GORE-TEX bags (Terrill P J, Kedwards S M, and Lawrence J C. (1991) The use of GORE-TEX bags for hand bums. Burns 17(2): 161-5), or other porous membranes that are coated with a material that prevents cellular adhesion, for example Teflon.

Gene products produced by micro-organs can then be delivered via, for example, polymeric devices designed for the controlled delivery compounds, e.g., drugs, including proteinaceous biopharmaceuticals. A variety of biocompatible polymers (including hydrogels), including both biodegradable and non-degradable polymers, can be used to form an implant for the sustained release of a gene product of the micro-organs in context of the invention at a particular target site. The generation of such implants is generally known in the art (see, for example, Concise Encyclopedia of Medical & Dental Materials, ed. By David Williams (MIT Press: Cambridge, Mass., 1990); Sabel et al. U.S. Pat. No. 4,883,666; Aebischer et al. U.S. Pat. No. 4,892,538; Aebischer et al. U.S. Pat. No. 5,106,627; Lim U.S. Pat. No. 4,391,909; and Sefton U.S. Pat. No. 4,353,888).

Production of the recombinant protein results in its local release and concurrent diffusion to the lymphatic system for ultimate systemic delivery.

Implantation of genetically modified micro-organs according to the present invention can be effected via standard surgical techniques or via injecting micro-organ preparations into the intended tissue regions of the mammal utilizing specially adapted syringes employing a needle of a gauge suitable for the administration of micro-organs.

Micro-organs may be implanted subcutaneously, intradermally, intramuscularly, intraperitoneally and intragastrically. In a preferred embodiment of the present invention, the donor micro-organs utilized for implantation are preferably prepared from an organ tissue of the recipient mammal, or a syngeneic mammal, although allogeneic and xenogeneic tissue can also be utilized for the preparation of the micro-organs providing measures are taken prior to, or during implantation, so as to avoid graft rejection and/or graft versus host disease (GVHD). Numerous methods for preventing or alleviating graft rejection or GVHD are known in the art and as such no further detail is given herein.

As used herein the term “donor” refers to the individual providing the explant tissue for processing into a biopump.

As used herein the term “recipient” refers to the individual being implanted with a biopump.

As used herein the term “syngeneic” refers to animal individuals, which are genetically similar.

As used herein the term “allogeneic” refers to animal individuals, which are genetically dissimilar but are from the same species

As used herein the term “xenogeneic” refers to animal individuals of different species.

As used herein, GVHD refers to graft versus host disease, a consequence of tissue transplantation (the graft) caused by the transplant immune response against the recipient host. More specifically, graft-versus-host disease is caused by donor T-lymphocytes (T cells), recognizing the recipient as being foreign and attacking cells of the recipient.

In another preferred embodiment of the present invention recipients include animal models such as, non-human primates, swine, such as wholly or partially inbred swine (e.g., miniature swine, and transgenic swine), rodents, sheep, dogs, cows, chickens, amphibians, reptiles, and mammals other than those listed herein.

In still another preferred embodiment the recombinant gene product may be produced continuously, or in response to an inducing signal. The product may cease being produced upon removal of the inducing agent. Examples of inducing agents commonly used to stimulate gene expression from appropriate promoters are isopropyl-beta-D-1-thiogalactopyranoside (IPTG), phorbol esters, hormones or metal ions, (Sassone-Corsi et al. (1986) Trends Genet. 2:215; Maniatis et al. (1987) Science 236:1237), and others.

Thus the preparation and implantation of the biopumps facilitates expression of a variety of recombinant protein-drug and functional RNA molecules within recipient animals, for subsequent functional analysis.

### Measuring Quantitative or Qualitative Pharmacological, Physiological and/or Therapeutic Parameters or Effects

The present invention provides a unique method for assessing a large array or parameters and effects, as a consequence of exposure to a recombinant gene product and represent preferred embodiments of the present invention. Included are a means of measuring pharmacological, pharmacokinetic, physiological, and therapeutic parameters and/or effects.

As used herein, the term “pharmacological” refers to the properties and reactions of drugs.

As used herein, the term “pharmacokinetic” refers to the action of drugs in the body over a period of time, including the processes of absorption, distribution, localization in tissues, biotransformation, and excretion.

As used herein, the term “physiological” refers to normal, not pathologic, characteristic of or conforming to the normal functioning or state of the body or a tissue or organ.

As used herein, the term “therapeutic” pertains to the art of healing, or curative.

As used herein, the term “efficacy” includes causing a desired functional or health state or condition to be achieved, or preventing or reducing the extent of an undesired health state or condition.

As used herein, the term “parameter” refers to a variable whose measure is indicative of a quantity or function that cannot itself be precisely determined by direct methods; e.g., blood pressure and pulse rate are parameters of cardiovascular function, and the level of glucose in blood and urine is a parameter of carbohydrate metabolism

As used herein, the term “effect” refers to the result produced by an action. In this case, effects are results of implantation of the biopumps, and elaboration of the recombinant gene product.

### Pharmacological Parameters or Effects

Biopumps may be utilized as a means of evaluating the pharmacological effects and parameters of a given recombinant gene product in vitro, and in vivo. Pharmacological effects, resulting from gene product elaboration from the biopumps, include both pharmacodynamic parameters and effects, i.e., where the drug localizes within the recipient, what the drug's activity is, and its mechanism of action, and pharmacokinetic parameters and effects, i.e. how the drug is metabolized in the recipient.

According to a preferred embodiment of the present invention, the pharmacodynamic parameter of recombinant gene product localization can be addressed by methods identifying both gene and protein expression, delineated above. Specific tissues may be isolated and homogenized, and nucleic acids/proteins analyzed for recombinant product expression, tissues may be processed, embedded and sectioned, or alternatively flash frozen and similarly evaluated. Circulating effects may be assessed by serum, plasma and/or lymph collection and similar analyses.

According to a preferred embodiment of the present invention, the pharmacodynamic parameter of recombinant gene product activity can be evaluated. If the recombinant gene product in question is, for example, an enzyme with a known enzymatic activity, a typical method for analysis would be enzyme-substrate analysis. If the recombinant gene product in question form a part of a ligand-receptor relationship, a ligand receptor analysis may be performed. Similarly, if the recombinant gene product stimulates cell proliferation, cellular differentiation/proliferation assays utilizing, for example, incorporation of radionucleotide labeled precursors may be utilized, and if the recombinant gene product is a proapoptotic stimulator, cell viability assays may be conducted. A variety of methods may be employed to assay recombinant protein activity, with the methods cited above to serve for exemplary purposes and should not be considered exclusive. Additionally, with any of the aforementioned methods, results obtained may be either quantitative (i.e., the numerical value obtained) or qualitative (e.g., detected or non-detected, implying a pre-set threshold of detection).

Biopumps provide a unique means to assess pharmacodynamic parameters and effects, as well. Recombinant gene products may be isolated, as may breakdown products, by the protein isolation or fractionation methods delineated above. Once isolated or fractionated, compositions may be assessed by a variety of methods well known in the art including, as indicated hereinabove, HPLC, mass spectroscopy, GLC, immunohistochemistry, ELISA, RIA, or western blot analysis.

### Physiological Parameters and Effects

Physiological parameters and effects of recombinant gene products may be readily assessed using biopumps. The term “physiological effect” encompasses effects produced in the subject that achieve the intended purpose of a treatment. In preferred embodiments, a physiological effect in a disease model means that the symptoms of the subject being treated are prevented or alleviated. For example, a physiological effect would be one that results in the prolongation of survival. Other examples of physiological effects compromise development of protective immune responses, immunity, cell proliferation, and other functions that contribute to the well-being, normal physiology, or general quality of life of the individual. Deleterious physiological effects may involve, but are not limited to, destructive invasion of tissues, growth at the expense of normal tissue function, irregular or suppressed biological activity, aggravation or suppression of an inflammatory or immunologic response, increased susceptibility to other pathogenic organisms or agents, and undesirable clinical symptoms such as pain, fever, nausea, fatigue, mood alterations, and other features.

Physiological parameters measured as an indication of specific physiological effects may include, but are not limited to, blood pressure, heart rate, fever, pain, plasma glucose, protein, urate/uric acid, carbonate, calcium, potassium, sodium, chloride, bicarbonate, glucose, urea, lactate/lactic acid, amylase, lipase, transaminase, billirubin, hydroxybutyrate, cholesterol, triglycerides, creatine, creatinine, pyruvic acid, TSH levels, hemoglobin and insulin levels, prostate specific antigen, hematocrit, blood gases concentration (carbon dioxide, oxygen, pH), lipid composition, electrolytes, iron, heavy metal concentration (e.g., lead, copper), and others. These parameters, in turn can be measured by the numerous assay systems discussed herein or otherwise well known in the art.

### Therapeutic Parameters or Effects

Therapeutic parameters and effects of recombinant gene products may be readily assessed using biopumps as well. Some of these effects include preventing occurrence or recurrence of disease, alleviation of symptoms, and diminishment of any direct or indirect pathological consequences of the disease, preventing metastasis, preventing death, decreasing the rate of disease progression, amelioration or palliation of the disease state, and remission or improved prognosis.

For in vivo analysis, implanted biopumps elaborate a given gene product and general therapeutic effects in the recipient animal can be evaluated, including, cytotoxicity of the candidate drug, organ toxicity, carcinogenicity, mutagenicity and teratogenicity.

As used herein the term “mutagenicity” refers to the induction of permanent transmissible changes in the amount or structure of genetic material of cells or organisms. These changes, “mutations”, may involve a single gene or gene segment, a block of genes, or whole chromosomes.

As used herein the term “carcinogenicity” refers to the induction of the disease cancer in any of its manifest phases including initiation, promotion and progression.

As used herein the term “teratogenicity” refers to the induction of processes resulting in fetal abnormalities.

As used herein the term “cytotoxicity” refers to the induction of cell death, mediated through either apoptotic or necrotic mechanisms of induction of cell death.

As used herein the term “organ toxicity” refers to induction of damage and cell death within cells of a particular organ.

Cytotoxicity may be assessed by vital staining techniques well known in the art. The effect of growth/regulatory factors may be assessed by analyzing the cellular content, e.g., by total cell counts, and differential cell counts. This may be accomplished using standard cytological and/or histological techniques including the use of immunocytochemical techniques employing antibodies that define type-specific cellular antigens. Similarly, organ toxicity can be assessed via macroscopic evaluation through a variety of techniques known to those skilled in the art including ultrasonography, computed tomography, magnetic resonance imaging and others. Lethal dose assessment and post-mortem pathological evaluation for gross anatomical changes may be conducted, assessing recombinant gene product toxicity.

In order to evaluate teratogenicity, pregnant female recipient animals may be utilized for implantation of the biopumps to facilitate evaluation of the candidate drug as a teratogen. Additional in vitro assays of teratogenicity may be performed including, but not limited to, assays utilizing embryonic cells obtained from rats and mice, as is well known in the art (Flint O. P. (1983) A micromass culture method for rat embryonic neural cells. J. Cell. Sci. 61: 247-262; Flint O. P. (1987) An in vitro test for teratogens using cultures of rat embryo cells. in In vitro Methods in Toxicology (eds. C. K. Atterwill and C. E. Steele) Cambridge University Press; Cambridge England, pp. 339-363; and Heuer J., Graeber I. M., Pohl I., and Spielmann H. (1994) An in vitro embryotoxicity assay using the differentiation of embryonic mouse stem cells into hematopoietic cells. Toxicol. In vitro 8: 558-587).

Finally, mutagenicity and carcinogenicity may be evaluated in vivo in distal sites within the recipient.

Determination of carcinogenicity may be a function of measuring cell proliferation. Such methods are well described in the art and most commonly include determining DNA synthesis characteristic of cell replication. There are numerous methods in the art for measuring DNA synthesis, any of which may be used according to the invention. In an embodiment of the invention, DNA synthesis can be determined using a radioactive label (3H-thymidine) or labeled nucleotide analogues (BrdU) for detection by immunofluorescence. Additional methods include evaluation of specific tumor-related events, such as the expression of any of a variety of known oncogenes, and the formation of detectable tumors.

Once a protein drug candidate has been evaluated in vivo for therapeutic efficacy using the methods of the present invention, mutagenicity may be determined as well via well-established protocols, including the bacterial reverse mutation or Ames assay, in vivo heritable germ cell mutagenicity assays (Waters M D, Stack H F, Jackson M A, Bridges B A, and Adler I D (1994). The performance of short-term tests in identifying potential germ cell mutagens: a qualitative and quantitative analysis. Mutat. Res. 341(2): 109-31) and in vivo somatic cell mutagenicity assays (Compton P J, Hooper K, and Smith M T. (1991) Human somatic mutation assays as biomarkers of carcinogenesis Environ Health Perspect 1991 August;94:135-41; Caspary W J, Daston D S, Myhr B C, Mitchell A D, Rudd C J, and Lee P S (1988) Evaluation of the L5178Y mouse lymphoma cell mutagenesis assay: inter-laboratory reproducibility and assessment. Environ. Mol. Mutagen. 12 Suppl 13:195-229; Wild D, Gocke E, Harnasch D, Kaiser G, and King M T (1985) Differential mutagenic activity of IQ (2-amino-3-methylimidazo[4,5-f]quinoline) in Salmonella typhimurium strains in vitro and in vivo, in Drosophila, and in mice. Mutat Res 156(1-2):93-102; and Holden H E (1982) Comparison of somatic and germ cell models for cytogenetic screening. .J Appl Toxicol 2(4): 196-200).

Hence, according to preferred embodiments of the present invention, pharmacokinetic, pharmacodynamic, physiologic and/or therapeutic parameters or effects of expressed recombinant proteins and/or protein-drugs may be measured in terms of efficacy, toxicity, mutagenicity, carcinogenicity and teratogenicity in vivo.

### Protein-Drug Optimization

Among the more difficult tasks in drug design is optimization of particular compounds once a therapeutic effect is discovered. Random testing in whole animals is a costly, time consuming procedure as outlined hereinabove. Generation of biopumps secreting various permutations of a particular recombinant protein enables the efficient evaluation of multiple recombinants, as well as enabling assessment of coincident synergistic or antagonistic effects.

Therefore, according to another embodiment of the present invention, there is provided a method of optimizing a protein-drug for determining pharmacological, physiological and/or therapeutic, quantitative or qualitative, parameters or effects. The method comprises providing a plurality of polynucleotides encoding recombinant gene products differing by at least one amino acid from the protein-drug; genetically modifying the micro-organ explants to express and secrete the proteins differing by the at least one amino acid, implanting them within recipients and comparing parameters or effects of the proteins differing by at least one amino acid with each other, and the protein drug in the recipient animal.

Implantation enables comparative determination of pharmacological, physiological and/or therapeutic, quantitative or qualitative, parameters or effects of the proteins for measurements in terms of efficacy, toxicity, mutagenicity, carcinogenicity and teratogenicity in vivo, as well. Simultaneous implantation within a single recipient of biopumps expressing different recombinant gene products enables the assessment of protein-drug synergistic or antagonistic effects, as well, and represents still additional preferred embodiments of the present invention.

### In vivo Functional Relationships Between Expressed Recombinant Gene Products

While multiple expressed recombinant gene products may interact competitively, or cooperatively with a singular mechanism of action, it is also to be envisaged that coordinate expression of two recombinant gene products may provide a means to assess functional relationships between the products in vivo.

In vitro assays addressing functional relationships between two proteins exist, but often rely upon physical proximity at a specified time for determination of cooperative activity. Chemical cross-linking of proteins, the yeast two hybrid system, and immunoprecipitation are the methods most commonly employed for determination of physical interactions between two proteins localized regionally. Functional relations are then often implied by juxtapositioning of the two proteins. Gene regulation effects by protein-nucleic acid interactions have also been demonstrated by gel mobility shift assays, revealing a functional relationship between specific proteins and nucleic acid sequences, and potentially, multiple proteins that may be involved.

These methods, however, do not address functional relationships between multiple proteins simultaneously, in vivo, in whole animal systems.

Hence, according to an aspect of the present invention there is provided a method of determining functional relations between recombinant gene products in vivo. The method according to this aspect of the invention comprises (a) providing at least one first polynucleotide encoding a first recombinant gene product; (b) providing at least one second polynucleotide encoding a second recombinant gene product whose expression potentially functionally modifies or regulates the expression and/or function of the first recombinant gene product; (c) obtaining a plurality of micro-organ explants from a donor subject, each of the plurality of micro-organ explants comprising a population of cells, each of the plurality of micro-organ explants maintaining a microarchitecture of an organ from which it is derived and at the same time having dimensions selected so as to allow diffusion of adequate nutrients and gases to cells in the micro-organ explants and diffusion of cellular waste out of the micro-organ explants so as to minimize cellular toxicity and concomitant death due to insufficient nutrition and accumulation of the waste in the micro-organ explants; (d) genetically modifying the plurality of micro-organ explants, so as to obtain a plurality of genetically modified micro-organ explants expressing and secreting the first and/or second recombinant gene products; (e) implanting the plurality of genetically modified micro-organ explants within a plurality of recipient subjects; and (f) determining the functional relations between the first and second recombinant gene products in vivo.

Functional relations between recombinant gene products may be determined at the level of RNA or protein expression or at the level of protein activity of one recombinant gene product in the presence and absence of the other recombinant gene product, via any of the methodologies listed hereinabove for evaluating RNA and/or protein expression or activity, and represent preferred embodiments of the present invention.

Comparative expression in this manner may elucidate a mechanism for the functional relationship between two or more recombinant gene products, in vivo.

Functional and/or structural modification and/or effects may include direct effects on the protein-protein interactions, such as effects on enzyme function, in for example, phosphorylation events, or in cleavage or alternate processing (such as glycosylation, phosphorylation, methylation or acetylation) of a protein to render it in its active form. Direct effects may also include functional assembly of protein complexes. Numerous methods are well known in the art for assessing these functional changes including specific assays of enzymatic activity, western blot analysis and immunohistochemistry probing with antibodies that specifically detect altered protein forms, including phosphorylated, methylated and glycosylated forms, and the assembly of protein complexes.

Functional and or structural modification and/or effects may also include indirect effects on protein-recombinant product interactions. Some preferred embodiments include the assessment of positive or negative effects exerted on promoter sequences, by functioning as a transacting factor, as, for example, an inducer, enhancer or suppressor, and these effects may be mediated in trans. The use of reporter constructs in the genetic modification of the biopumps may facilitate ready identification of these indirect effects, and as such comprise a preferred embodiment of the present invention. These effected changes may be measured by methods disclosed hereinabove, including PCR, RT-PCR, Northern blot analysis, nuclear run-on assays and gel mobility shift assays.

### In Vitro-In vivo Correlation Models for Recombinant Gene Product/Protein Drug Dosage and Function

Both in vitro and in vivo methods may be employed to assess the pharmacologic, physiologic and therapeutic parameters and effects discussed. Moreover, in a preferred embodiment of the present invention there is therefore provided a method of establishing an in vitro-in vivo correlation model, wherein prior to implanting biopumps into a recipient animal, an in vitro secretion level of the recombinant gene product is determined and, following implantation a corresponding in vivo level is determined, and the results compared to provide a meaningful, statistically evaluated result.

An example of an in vitro-in vivo correlation model may be the evaluation of the production of a cytokine. In vitro analysis via ELISA of micro-organ supernatants provides a value for the concentration of the cytokine produced by the micro-organs, as a function of time in culture. Once implanted, circulating levels of cytokine may be similarly assessed by ELISA assay of serum collected from implanted animals. A correlation between the values obtained for the cytokine production in both systems will provide information that reflects micro-organ production in vivo, and cytokine stability. One application of this model would be the extrapolation of the amount of production required in vitro for sufficient, sustained release in vivo, in constructing the biopumps. Similarly, many other models may benefit from in vitro-in vivo correlation data for optimization of dosage and effects of expressed recombinant products.

In terms of treatment, a drug effective amount can be ascertained in this system as well, and represents yet another preferred embodiment of the present invention. The effective amount is the amount that is sufficient to palliate, ameliorate, stabilize, reverse or slow the progression of the disease, or otherwise reduce the pathological consequences of a disease.

### Animal Models of Disease

Pharmacologic, physiologic and therapeutic parameters and effects may be evaluated in vivo in established animal models of disease. These models may include animal models for the study of:

Diabetes, both types I and II, employing the NOD mice, Ob mice, Db mice, BB rats, Wistar furry rats and obese Zucker diabetic fatty (ZDF-drt) rats (Jiao, S.; Matsuzawa, Y.; Matsubara, K.; Kubo, M.; Tokunaga, K.; Odaka, H.; Ikeda, H.; Matsuo, T.; Tarui, S. And Basingstoke, A. (1991) A new genetically obese rat with non-insulin-dependent diabetes mellitus (Wistar fatty rat). International journal of obesity v. 15 (7): p. 487-495; Lee, Y (1994) Obese Zucker diabetic fatty (ZDF-drt) rats. Proceedings of the National Academy of Sciences of the United States of America v. 91 (23): p. 10878-10882; Velliquette RA et al. (2002) Obese spontaneous hypertensive rat (SHROB), a unique animal model of leptin resistance and metabolic Syndrome X. Exp Biol Med 227(3): 164-70; and Scott J. (1990) The spontaneously diabetic BB rat: sites of the defects leading to autoimmunity and diabetes mellitus. A review. Curr Top Microbiol Immunol 156:1-14), and others.

Cardiovascular disease, employing the ischemia/reperfusion model (HR Cross (2002) Cardiovasc Res. 53(3):662-71), isoproterenol-induced myocardial infarction model (Arteaga de Murphy C (2002) Int J Pharm. 233(1-2):29-34), ligation induced myocardial infarction model (Bollano E. (2001) Eur J Heart Fail. 3(6):651-60.), and others.

Renal disease, employing the spontaneous nephrotic ICGN mice, (Ogura, A.; Asano, T.; Matsuda, J.; Takano, K; Nakagwa, M.; and Fukui, M. (1989) London: Royal Society of Medicine Services; 1989 April Laboratory animals v. 23 (2): p. 169-174), and others.

Alzheimer's disease, employing mouse strains with mutations in presenilin genes (Chui D-H, Tanahashi H, Ozawa K, Ikeda S, Checler F, Ueda O, Suzuki H, Araki W, Inoue H, Shirotani K, Takahashi K, Gallyas F, and Tabira T. (199) Aged transgenic mice carrying Alzheimer's presenilin 1 mutations show accelerated neurodegeneration without amyloid plaque formation. Nature Medicine 5: 560-564; Shirotani K, Takahashi K, Araki W, Tabira T. (2000) Mutational analysis of intrinsic regions of presenilin 2 which determine its endoproteolytic cleavage and pathological function. J Biol Chem 275(5):3681-6), and others.

Cancer, employing animal species with a high level of spontaneous tumor formation including dog and cat species (Vail D M, (2000) Cancer Invest 18(8):781-92), and rodents (Radl J.(1999) J.Pathol Biol 47(2):109-14; Martens A C (1990) Leukemia 4(4):241-57; Zevenbergen, J. L.; Verschuren, P. M.; Zaalberg, J.; Stratum, P. van; Vles, R. O. (1992). Nutrition and cancer v. 17 (1): 9-18); tumor cell injection in nude mice or rats (Schabet M (1998) J Neurooncol 38(2-3): 199-205); radiation induced melanomas (Corominas M (1991) Environ Health Perspect 93: 19-25), oncogene transgenic mice (Willems L (2000) AIDS Res Hum Retroviruses 16(16):1787-95), chronic viral induced carcinogenesis (Tennant BC (2001) ILAR J 42(2):89-102), and numerous other mice transgenic for targeted mutations in specific oncogenes and/or tumor suppressor genes.

Additional models including animal models of infection, autoimmune disorders, cystic fibrosis, muscular dystrophy and osteoporosis may be envisioned, as well as alternative models for the diseases listed hereinabove. These models are represented by way of exemplification alone, and are not intended to be exclusionary. In vitro disease models may similarly be evaluated, as well.

Thus according to additional preferred embodiments of the present invention, determining at least one pharmacological, physiological and/or therapeutic, quantitative or qualitative, parameters or effects of the recombinant gene product in the animal include determining animal survival and/or animal pathogen burden within at least one organ, in normal or diseased mice, including any of the models disclosed hereinabove, or others.

Comparative evaluation of animals implanted with different recombinant gene products, differing as indicated above by a single amino acid, for protein-drug optimization efforts, may similarly be evaluated, in terms of relative animal survival and/or animal pathogen burden and represents still other preferred embodiments of the present invention.

### Limitations of Gene Therapy

Applications of in vivo introduction of genetic sequences for in vivo production of recombinant gene products, (and in cases where the construct provides for the production of a product that is otherwise defective or absent the methodology is otherwise referred to as “gene therapy”), have significant limitations.

Gene therapy attempts have utilized retrovirus-based vectors, yet these vectors must integrate into the genome of the target tissue to allow for transgene expression (with the potential to activate resident oncogenes) while vector titers produced in such systems are significantly less than in some other systems. Because of the requirement for integration into the subject genome, the retrovirus vector can only be used to transduce actively dividing tissues, posing another limitation to the method application. Further, many retroviruses have limited host tissue specificity and cannot be employed to transduce more than a few specific tissues of the subject (Kurian K M, Watson C J, Wyllie A H. (2000) Mol Pathol. 53(4):173-6).

Adenoviral vectors have been another preferred vector of choice for gene therapy attempts, but they too are limited in potential therapeutic use for several reasons. First, due to the size of the El deletion and to physical virus packaging constraints, first generation adenovirus vectors are limited to carrying approximately 8.0 kb of transgene genetic material. While this compares favorably with other viral vector systems, it limits the usefulness of the vector where a larger transgene is required. Second, infection of the E1-deleted first generation vector into packaging cell lines leads to the generation of some replication competent adenovirus particles, because only a single recombination event between the E1 sequences resident in the packaging cell line and the adenovirus vector genome can generate a wild-type virus. Therefore, first-generation adenovirus vectors pose a significant threat of contamination of the adenovirus vector stocks with significant quantities of replication competent wild-type virus particles, which may result in toxic side effects if administered to a gene therapy subject (Rubanyi, G. M. (2001) Mol Aspects Med 22(3): 113-42.

The most difficult problem with most vectors employed, including adenovirus vectors is their inability to sustain long-term transgene expression, secondary to host immune responses that eliminate virally transduced cells in immune-competent animals (Gilgenkrantz et al., (1995) Hum. Gene Ther. 6:1265-1274; Yang et al., (1995) J. Virol. 69:2004-2015; Yang et al., (1994) Proc. Natl. Acad. Sci. USA 91:44074411; Yang et al., (1995) J. Immunol. 155: 2565-2570). It has also been clearly demonstrated that vector epitopes are major factors in triggering the host immune response (Gilgenkrantz et al., (1995) Hum. Gene Ther. 6:1265-1274; Yang et al., (1996) J. Virol. 70: 7209-7212). Recombinant protein introduction by methods disclosed in the present invention is therefore a superior technology for a number of reasons.

Unlike retroviral vectors, which provide limited organ tropism for site-specific product expression, biopumps can be implanted in numerous sites in the body. Integration-related issues are completely avoided, as is the necessity for actively dividing tissue for uptake of the construct. Large transgenes can be introduced into the biopumps, and contamination events avoided. Furthermore, as described in one of the preferred embodiments of the present invention, biopumps may be encased in a membranous packaging facilitating product export, but preventing immune cells and their secreted products from entering the biopump, and abrogating production, thereby extending the length of time the recombinant product is produced.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

In the following examples the method for recombinant gene product expression from implantable genetically modified micro-organs, or biopumps, has been shown to be stable, long term, and provide for sustained release of the recombinant product, in vivo.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are well known in the art and are provided for the convenience of the reader. All the information contained therein is incorporated herein by reference.

### Example 1

In Vitro Micro-Organ Expression of Murine Erythropoietin

Material and Experimental Methods

Preparation of Human Skin Micro-Organs

Approval for experiments utilizing human skin was obtained from the Rambam Hospital, Israel, according to standards approved by the Helsinki committee. A section of 1.4-1.5 mm human female skin thickness (depth) was aseptically removed from the abdomen according to standard operating procedures. The biopsy tissue was treated with a hypochloride solution (10% Milton solution), for 7 minutes followed by 3 washes with 20 ml DMEM for 10 minutes each. Following treatment, the tissue was further sectioned with a tissue chopper (TC-2 chopper, Sorval, Du-pont instruments). Tissue sectioning into 300 μm width explants was conducted under sterile conditions. The resulting micro-organs (MOs) were placed individually within wells of a 48-well micro-plate containing 400 μl per well of DMEM (Biological Industries —Beit Haemek) in the absence of serum under 5% CO² at 37° C. for 24 hours.

Preparation of Murine Skin and Lung Micro-Organs:

Lung or skin tissue of C-57B1/6 mice were excised, cleaned of debris, washed 3-4 times using DMEM, (Biological Industries Co., Beit Haemek) supplemented with L-glutamine and a solution of Penicillin/Streptomycin (stock 1000 u/ml, 100 mg/ml; diluted 1:100; Biological Industries, Co., Beit Haemek) [herein referred to as DME-C] in 90 mm Petri dishes and kept on ice. Lung and skin tissues were then cut into 300 μm sections (TC-2 tissue sectioning, Sorval Du-pont instruments), creating MOs. MOs were washed 3 times with DMEM, and 15 MOs were placed within each well of 48 multi-well plates, with 300 μl of DME-C.

MO Transfection with pORF-EF1a/hEPO-plasmid:

Human skin MO's were transfected with the commercially available pORF-hEPO-plasmid vector (porf-hepo-200, In-vivo Gene, San Diego, Calif. USA) using the Lipofectamine 2000 reagent (Life Technologies, Cat. No. 11668-027) according to manufacturer's instructions, with modifications as follows:

Prior to transfection with plasmid DNA, MO's were pulsed with 5 mM CaCl₂ for 1 hr, at 37° C. (5% CO2) with agitation. Endogenous DNases were inactivated using aurintricarboxylic acid (ATA substance) (Sigma, Cat. No. A5206) which was added to achieve a final concentration range of 1 or 10 ng/ml.

2.5 μl LF-2000 (Life Technologies) was diluted into 50 μl Opti-MEM (Life Technologies) and incubated at room temperature for 5 minutes, followed by the addition of log of DNA (pORF-hEPO) diluted into 50 μl Opti-MEM. The solution was incubated for 20 minutes at room temperature, and 100 μl of the complexes were added to each well (24-well plate) containing 5 MO's in 500 μl DMEM. The MOs were incubated for 24 hours at 37° C. in 5% CO₂, and media was replaced, then collected and changed every three days.

Centrifugation effects on transfection efficiency were analyzed by including a sample with transfected MO's centrifuged immediately after the addition of the plasmid, at 2000 rpm for 30 minutes in a 24 well plate. Samples of the culture medium containing pORF-EF1a/hEPO transformed biopumps were analyzed for hEPO secretion levels using an ELISA kit for HEPO. (Quantikine, IVD, R&D systems)

Micro-Organ Ttransduction with AAV2-CMV/mEPO:

The commercially available vector comprising the adeno-associated virus expressing murine erythropoietin off the cytomegaloviral promoter (designated AAV2-CMV/mEPO) was purchased from Genethon (center for research and application on gene therapies, Evry Cedex, France.)

Transduction of micro-organs was accomplished as follows: Two doses of adeno-associated virus [AAV] containing murine erythropoietin cDNA were transduced into the above-prepared MOs. Viral titers utilized for micro-organ infection were 3×10⁸ infective particles (IP)/ml and 3×10⁹ IP/ml. MOs were transduced with the viral vectors for 24 hours at 37° C. in an atmosphere of 5% CO2. Excess viral particles were removed by washing the wells three times with DMEM. Medium including the secreted mEPO was collected at 4, 7, 11 and 14 days post transduction.

Assessment of in vitro Protein Production:

Media was removed from each well every 2-3 days and assayed via ELISA for the presence of secreted mEPO (Quantikine, IVD, R&D systems). Cultures were replenished with media, accordingly.

Experimental Results

Micro-organs incorporate and express murine erythropoietin and secrete high levels of the protein for prolonged time periods in vitro

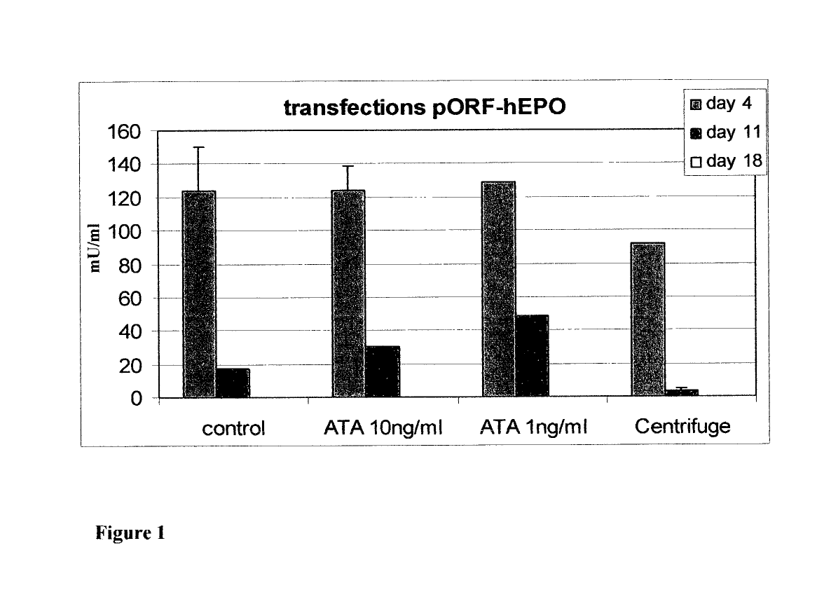

Human skin MO transfection with plasmid DNA encoding pORF-hEPO enabled efficient transgene expression using any of the various transfection protocols, all yielding similar results (FIG. 1), and inactivation of endogenous DNases prior to transfection facilitated longer maintenance of transgene expression, even 11 days post transfection. Centrifugation provided little positive effect and perhaps hampered transgene incorporation efficiencies, with transgene expression absent by 18 days post transfection.

Incorporation of mEPO via human skin MO transduction with the AAV2-CMV/mEPO construct provided for prolonged production and secretion of the transduced mEPO product. In vitro secretion levels of mEPO from human skin MOs transduced with the AAV2-CMV/mEPO construct were analyzed using a human ELISA kit. Since a commercial ELISA kit for mouse EPO is not available, we used a human EPO ELISA kit for the analysis, which detected murine EPO, as well. As a consequence, however, the units on the Y-axis are arbitrary units.

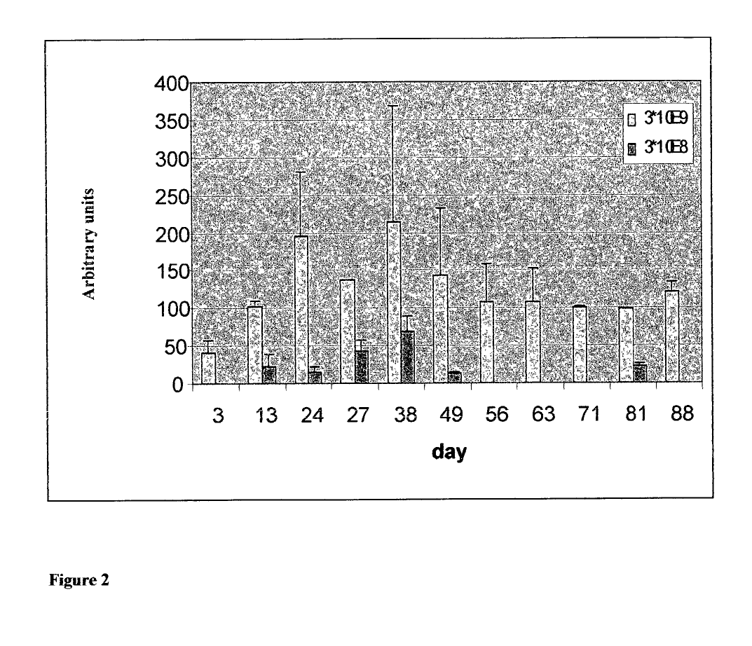

Significantly, human skin biopumps secreted the desired EPO protein in vitro for as long as 88 days, as compared to controls (FIG. 2). Secretion was dose dependant, as MOs transduced with 3×10⁹ IP/ml gave significantly higher secretion levels as compared to MOs transduced with 3×10⁸ IP/ml and controls.

### Example 2

In vivo Micro-Organ Expression of Murine Interferon-α

Material and Experimental Methods

Construct Preparation:

The commercially available vector comprising strain 5 of the adenovirus expressing murine interferon a off the cytomegaloviral promoter (designated Ad5-CMV/mIFNα) and a vector comprising strain 5 of the adenovirus expressing the β-galactosidase gene, (designated Ad5-CMV/LacZ), used as a control, were both purchased from Q-Biogene (Carisbad, Calif., USA).

Ad5-CMV/mIFN α micro-organ implantation:

Male and female SCID mice weighing around 25 grams were anaesthetized with 140 ul of diluted Ketast (ketamine HCl) (400 μl Ketast and 600 μl saline) and Ad5-CMV/mIFN α expressing MOs were implanted subcutaneously, 14 days following MO transduction.

Assessment of in vitro Protein Production:

Media was removed from each well every 2-3 days and assayed via ELISA for the presence of secreted mIFNα (Cell Science Inc., Cat. No. CK 2010-Norwood Mass., USA.). Cultures were replenished with media, accordingly.

Assessment of in vivo Protein Production:

Serum was collected via bleeding trough the eye according to standard procedures on days 6, 16, 27, 55, 69, and 111 post-implantation of the micro-organs. Serum was diluted 1:2, with kit dilution buffer and assayed via ELISA for the presence of secreted mIFNα (Science Inc., Cat. No. CK 2010-1Norwood Mass., USA).

Assessment of in vitro- in vivo correlation of protein production:

In vitro production of mIFNα was tabulated as a function of the number of nanograms of protein produced as a function of time, per micro-organ cultured 10 (ng/day/MO). -In vivo production of mIFNα was tabulated as a function of the number of picograms of protein detected per ml of blood collected following implantation. The data were then correlated directly and plotted.

Viral Cytopathic Inhibition Assay:

1×10⁴ LTK cells were plated in DMEM containing 10% fetal calf serum (FCS). 24 hours later the medium was removed and replaced with 50 ul DMEM containing 2% FCS. In addition 4 or 8 ul of serum collected from mice implanted with Ad5-CMV/mIFNα biopumps were added to each well. As a control, a known concentration (U/ml) of recombinant mIFNα in DMEM containing 10% FCS, was added to a different set of wells, and served as the standard curve.

After 24 hours in culture, vesicular stomatitis virus (VSV) was added to all wells in a volume of 100 ul, at mode of infection (MOI) of 10:1, cells:virus, respectively, and incubated for an additional 24 hours. An MTT (4,5, dimethylthiaazol 2-yl-2,5, diphenyl tetrazolium bromide) assay measuring cell viability as a function of OD was performed in which the level of the IFNα anti-cytopathic effect in response to VSV infection was estimated according to the OD measurements obtained in the MTT assay.

All other procedures including preparation of human skin micro-organs and micro-organ transduction were conducted as in example 1, with the appropriate constructs being substituted for the present application.

Experimental Results

Implanted MOs Expressing Murine Interferon Alpha Secrete High in vivo Levels of the Expressed Protein

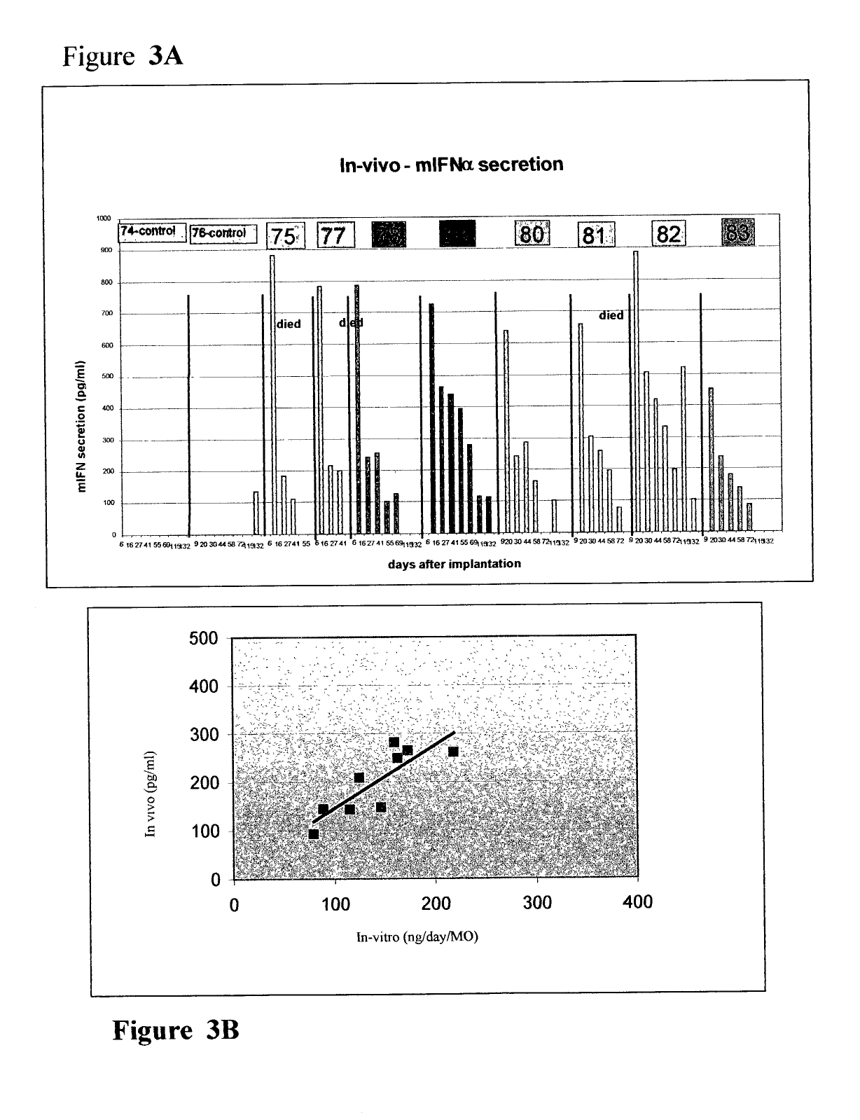

Human skin micro-organs were prepared as described above and transduced with an adenoviral vector expressing the gene for mouse interferon alpha (Ad5-CMV/mIFNα). MOs expressing mIFNα were implanted sub-cutaneously in 8 SCID mice while control mice were implanted with MOs transduced with a similar construct expressing the lacZ gene (Adeno-lacZ). Serum was then assayed for mIFNα presence on the days specified. Mice implanted with Ad5-CMV/mIFNα MOs revealed elevated serum levels of mIFNα, as compared to controls, at the indicated time points (FIG. 3A). Most surprisingly, in vivo mIFNα, production correlated directly with in vitro MO production (FIG. 3B). These data indicated that in vitro secretion levels, measured prior to implantation, were predictive for in vivo circulating levels, herein determined. Thus, in vitro secretion levels may be used to determine the amount of biopump that should be implanted back into a patient, to achieve desired circulating levels of any given protein.

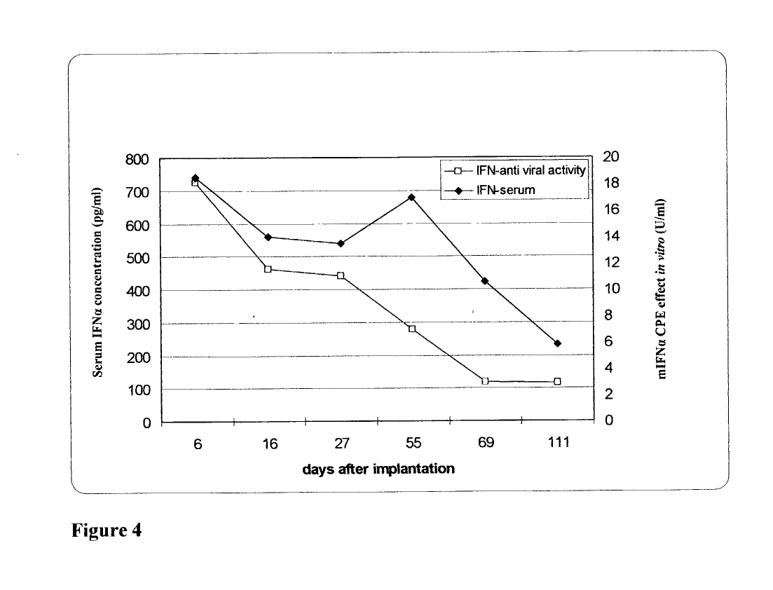

The secreted mIFNα was biologically active, as determined by viral cytopathic inhibition assay (FIG. 4). Viral cytopathic activity almost directly paralleled that of mIFNα circulating levels, indicating a causal relationship between the two.

### Example 3

Implanted Microorgans Maintain Structural Integrity Over Time

Material and Experimental Methods

Preparation of Murine Lung Micro-Organs:

Entire lungs were removed from several C57B1/6 mice and then lower right or left lobes of the lungs were aseptically dissected. The tissue was further sectioned with a tissue chopper (TC-2 Tissue sectioning, Sorval Du-pont instruments) into 300 μm width explants, under sterile conditions. The resulting micro-organs (MOs) were placed within wells of a 48-well micro-plate containing 400 μl of DMEM (Biological Industries—Beit Haemek) in the absence of serum, per well, and incubated under a 5% CO2 atmosphere, at 37° C. for 24 hours. Wells were visualized under a binocular (Nikon-SMZ 800) microscope and micro-organs were photographed, accordingly.

Experimental Results

MOs Maintain Macroscopic Integrity during Long-Term Sub-Cutaneous Implantation

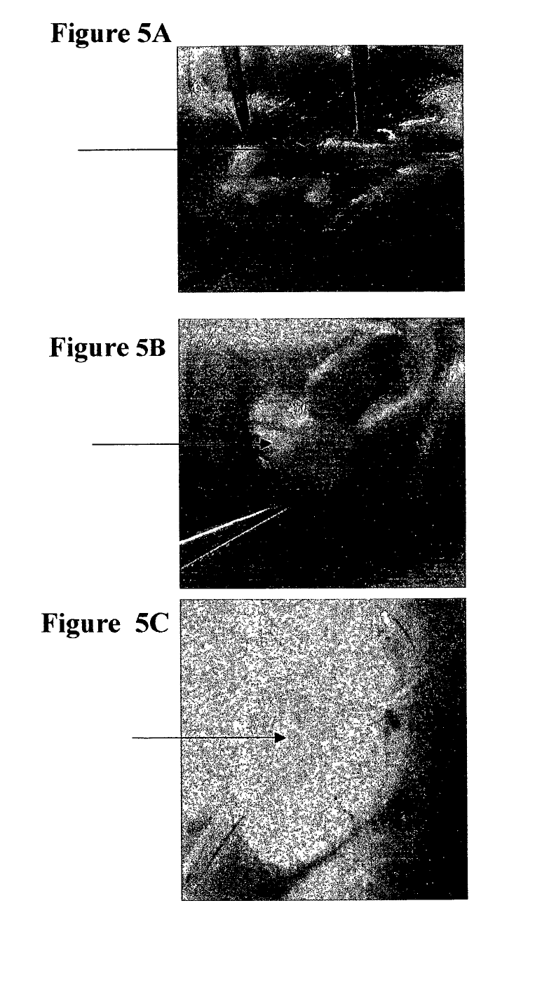

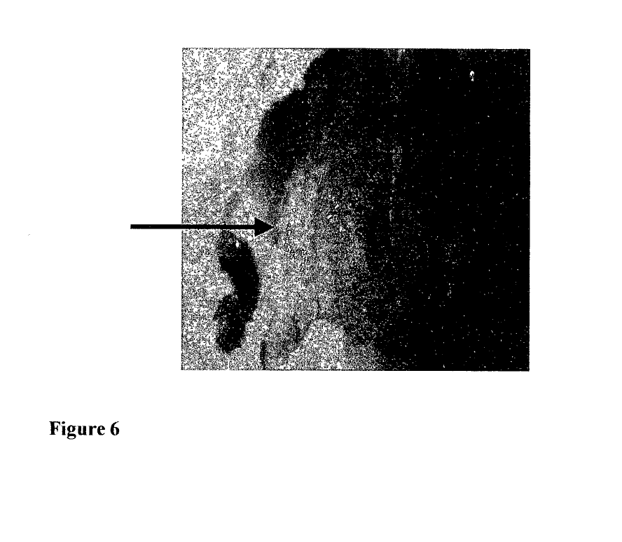

Mouse lung MO's were prepared similarly to human skin MOs described above, and implanted sub-cutaneously in normal syngeneic immunocompetent C57B1/6 mice (mouse lung MOs), or in SCID mice (human skin MOs). Lung MO's maintained structural integrity even 140 (A & B), and 174 (C) days post-implantation (FIG. 5A, FIG. 5B and FIG. 5C). Similarly, human skin biopumps maintained structural integrity as long as 76 days post-implantation within SCID mice (FIG. 6).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications mentioned in this specification are herein incorporated in their entirety by reference into the specification, to the same extent as if each individual publication, patent, patent application was specifically and individually indicated to be incorporated herein by reference. In addition, citation or identification of any reference in this application shall not be construed as an admission that such reference is available as prior art to the present invention.

What is claimed is:

1. A method of determining at least one quantitative or qualitative pharmacological, physiological and/or therapeutic, parameter or effect of a recombinant gene product in vivo, the method comprising:
(a) obtaining at least one micro-organ explant from a donor subject, said micro-organ explant comprising a population of cells, said micro-organ explant maintaining a microarchitecture of an organ from which it is derived and at the same time having dimensions selected so as to allow diffusion of adequate nutrients and gases to cells in said micro-organ explant and diffusion of cellular waste out of said micro-organ explant so as to minimize cellular toxicity and concomitant death due to insufficient nutrition and accumulation of said waste in said micro-organ explant, at least some cells of said population of cells of said micro-organ explant expressing and secreting at least one recombinant gene product;
(b) implanting said at least one micro-organ explant in a recipient subject; and
(c) determining said at least one quantitative or qualitative pharmacological, physiological and/or therapeutic, parameter or effect of said recombinant gene product in said recipient subject.

2. The method of claim 1, wherein said recombinant gene product is encoded by an expressed sequence tag (EST).

3. The method of claim 1, wherein said recombinant gene product is of an unknown function.

4. The method of claim 1, wherein said recombinant gene product is of a known function.

5. The method of claim 1, wherein said recombinant gene product is of a suspected function.

6. The method of claim 1, wherein said recombinant gene product is of a suspected function based on sequence similarity to a protein of a known function.

7. The method of claim 1, wherein said recombinant gene product is encoded by a polynucleotide having a modified nucleotide sequence as compared to a corresponding natural polynucleotide.

8. The method of claim 1, wherein said cells of said micro-organ explant expressing and secreting said at least one recombinant gene product are a result of genetic modification of at least a portion of the population of cells by transfection with a recombinant virus carrying a recombinant gene encoding said recombinant gene product.

9. The method of claim 8, wherein said recombinant virus is selected from the group consisting of a recombinant hepatitis virus, a recombinant adenovirus, a recombinant adeno-associated virus, a recombinant papilloma virus, a recombinant retrovirus, a recombinant cytomegalovirus, a recombinant simian virus, a recombinant lenti virus and a recombinant herpes simplex virus.

10. The method of claim 1, wherein said cells of said micro-organ explant expressing and secreting said at least one recombinant gene product are transduced with a foreign nucleic acid sequence via a transduction method selected from the group consisting of calcium-phosphate mediated transfection, DEAE-dextran mediated transfection, electroporation, liposome-mediated transfection, direct injection, gene gun transduction, pressure enhanced uptake of DNA and receptor-mediated uptake.

11. The method of claim 1, wherein said cells of said micro-organ explant expressing and secreting said at least one recombinant gene product are a result of genetic modification of at least a portion of the population of cells by uptake of a non-viral vector carrying a recombinant gene encoding said recombinant gene product.

12. The method of claim 11, wherein said cells are transduced with a foreign nucleic acid sequence via a transduction method selected from the group consisting of calcium-phosphate mediated transfection, DEAE-dextran mediated transfection, electroporation, liposome-mediated transfection, direct injection, gene gun transduction, pressure enhanced uptake of DNA and receptor-mediated uptake.

13. The method of claim 1, wherein said recombinant gene product is under a control of an inducible promoter.

14. The method of claim 1, wherein said recombinant gene product is under a control of a constitutive promoter.

15. The method of claim 1, wherein said recombinant gene product is selected from the group consisting of a recombinant protein and a recombinant functional RNA molecule.

16. The method of claim 1, wherein said recombinant gene product is normally produced by the organ from which the micro-organ explant is derived.

17. The method of claim 1, wherein said recombinant gene product is normally not produced by the organ from which the micro-organ explant is derived.

18. The method of claim 1, wherein said recombinant gene product is encoded with a known tag peptide sequence to be introduced into the recombinant protein.

19. The method of claim 1, wherein said recombinant gene product is encoded with a polycistronic recombinant nucleic acid including an IRES site sequence, a sequence encoding a reporter protein, and a sequence encoding the protein of interest.

20. The method of claim 1, wherein said recombinant gene product comprises a marker protein.

21. The method of claim 1, wherein said recombinant gene product is selected from the group consisting of insulin, amylase, a protease, a lipase, a kinase, a phosphatase, a glycosyl transferase, trypsinogen, chymotrypsinogen, a carboxypeptidase, a hormone, a ribonuclease, a deoxyribonuclease, a triacylglycerol lipase, phospholipase A2, elastase, amylase, a blood clotting factor, UDP glucuronyl transferase, ornithine transcarbamoylase, cytochrome p450 enzyme, adenosine deaminase, serum thymic factor, thymic humoral factor, thymopoietin, a growth hormone, a somatomedin, a costimulatory factor, an antibody, a colony stimulating factor, erythropoietin, epidermal growth factor, hepatic erythropoietic factor (hepatopoietin), a liver-cell growth factor, an interleukin, an interferon, a negative growth factor, a fibroblast growth factor, a transforming growth factor of the α family, a transforming growth factor of the β family, gastrin, secretin, cholecystokinin, somatostatin, substance P, a ribozyme and a transcription factor.

22. The method of claim 1, wherein said micro-organ explant is immune-protected by a biocompatible immuno-protective sheath.

23. The method of claim 1, wherein said at least one pharmacological, physiological and/or therapeutic effect comprises efficacy.

24. The method of claim 1, wherein said at least one pharmacological, physiological and/or therapeutic effect comprises toxicity.

25. The method of claim 1, wherein said at least one pharmacological, physiological and/or therapeutic effect comprises mutagenicity.

26. The method of claim 1, wherein said at least one pharmacological, physiological and/or therapeutic effect comprises carcinogenicity.

27. The method of claim 1, wherein said at least one pharmacological, physiological and/or therapeutic effect comprises teratogenicity.

28. The method of claim 1, wherein said recipient subject is an established animal model for a human disease.

29. The method of claim 1, wherein prior to said implanting, an in vitro secretion level of said gene product is determined.

30. The method of claim 29, wherein prior to said step of implanting, an in vitro secretion level of said gene product from said micro-organ is determined and an in vitro-in vivo correlation model is constructed for said animal model, so as to enable quantitative prediction and adjustment of the expression level in said animal model.

31. The method of claim 1, used for determining an in vivo effect of a protein-based drug.

32. The method of claim 1, used for analyzing at least one pharmacokinetic parameter of a protein-based drug in vivo.

33. The method of claim 1, used for analyzing at least one pharmacodynamic parameter of a protein-based drug in vivo.

34. The method of claim 1, used for analyzing at least one physiologic parameter of a protein-based drug for in vivo.

35. The method of claim 1, used for analyzing at least one therapeutic parameter of a protein-based drug for in vivo

36. The method of claim 1, used for analyzing efficacy of a protein-based drug in vivo.

37. The method of claim 1, used for analyzing toxicity of a protein-based drug in vivo.

38. The method of claim 1, used for analyzing mutagenicity of a protein-based drug in vivo.

39. The method of claim 1, used for analyzing carcinogenicity of a protein-based drug in vivo.

40. The method of claim 1, used for analyzing teratogenicity of a protein-based drug in vivo.

41. The method claim 1, wherein said dimensions are selected such that cells positioned deepest within said micro-organ explant are at least about 125-150 micrometers and not more than about 225-250 micrometers away from a nearest surface of said micro-organ explant.

42. The method of claim 41, wherein said organ is selected from the group consisting of a lymph system organ, a pancreas, a liver, a gallbladder, a kidney, a digestive tract organ, a respiratory tract organ, a reproductive system organ, skin, a urinary tract organ, a blood-associated organ, a thymus and a spleen.

43. The method of claim 41, wherein said micro-organ explant comprises epithelial and connective tissue cells, arranged in a microarchitecture similar to the microarchitecture of the organ from which the explant was obtained.

44. The method of claim 41, wherein the organ is a pancreas and the population of cells comprise islets of Langerhan.

45. The method of claim 41, wherein the organ is skin and the explant comprise at least one hair follicle and at least one gland.

46. The method of claim 41, wherein the organ is a diseased tissue, and the explant comprises a population of hyperproliferative or neoproliferative cells from the diseased tissue.

47. The method of claim 41, wherein the organ is a normal tissue.

48. The method of claim 1, wherein the organ is a normal tissue.

49. The method of claim 1, wherein said micro-organ explant has a surface area to volume index characterized by the formula 1/x+1/a>1.5 mm-1; wherein ‘x’ is a tissue thickness and ‘a’ is a width of said tissue in millimeters.

50. The method of claim 49, wherein said organ is selected from the group consisting of a lymph organ, a pancreas, a liver, a gallbladder, a kidney, a digestive tract organ, a respiratory tract organ, a reproductive organ, skin, a urinary tract organ, a blood-associated organ, a thymus and a spleen.

51. The method of claim 49, wherein said micro-organ explant comprises epithelial and connective tissue cells, arranged in a microarchitecture similar to the microarchitecture of the organ from which the explant was obtained.

52. The method of claim 49, wherein the organ is a pancreas and the population of cells comprise islets of Langerhan.

53. The method of claim 49, wherein the organ is skin and the explant comprise at least one hair follicle and at least one gland.

54. The method of claim 49, wherein the organ is a diseased tissue, and the explant comprises a population of hyperproliferative or neoproliferative cells from the diseased tissue.

55. The method of claim 1, wherein said micro-organ explant is derived from the recipient subject.

56. The method of claim 1, wherein said donor subject is a human being.

57. The method of claim 1, wherein said donor subject is a non-human animal.

58. The method of claim 1, wherein said recipient subject is a human being.

59. The method of claim 1, wherein said recipient subject is a non-human animal.

60. The method of claim 1, wherein said at least some cells of said population of cells of said micro-organ explant express and secrete said at least one recombinant gene product in a continuous, sustained fashion.

61. The method of claim 1, wherein said at least some cells of said population of cells of said micro-organ explant express and secrete said at least one recombinant gene product in a continuous, sustained fashion, following administration of an inducing agent.

62. The method of claim 61, wherein said at least some cells of said population of cells of said micro-organ explant cease to express and secrete said at least one recombinant gene product, following administration of a repressor agent.

63. The method of claim 61, wherein said at least some cells of said population of cells of said micro-organ explant cease to express and secrete said at least one recombinant gene product, following removal of said inducing agent.

64. The method of claim 1, wherein determining said at least one quantitative or qualitative pharmacological, physiological and/or therapeutic parameter or effect of said recombinant gene product in said recipient subject comprises determining survival.

65. The method of claim 1, wherein determining said at least one quantitative or qualitative pharmacological, physiological and/or therapeutic parameter or effect of said recombinant gene product in said recipient subject comprises determining apoptosis and necrosis.

66. The method of claim 1, wherein determining said at least one quantitative or qualitative pharmacological, physiological and/or therapeutic, parameter or effect of said recombinant gene product in said recipient subject comprises determining pathogen burden within at least one organ.

67. The method of claim 1, wherein determining said at least one quantitative or qualitative pharmacological, physiological and/or therapeutic, parameter or effect of said recombinant gene product in said recipient subject comprises using at least one of the following assays: ELISA, Western blot analysis, HPLC, mass spectroscopy, GLC, immunohistochemistry, RIA, metabolic studies, patch-clamp analysis, perfusion assays, PCR, RT-PCR, Northern blot analysis, Southern blot analysis, RFLP analysis, nuclear run-on assays, gene mapping, cell proliferation assays and cell death assays.

68. A method of optimizing a protein-drug comprising:
(a) providing a plurality of polynucleotides encoding recombinant gene products differing by at least one amino acid from the protein-drug;
(b) obtaining a plurality of micro-organ explants from a donor subject, each of said plurality of micro-organ explants comprises a population of cells, each of said plurality of micro-organ explants maintaining a microarchitecture of an organ from which it is derived and at the same time having dimensions selected so as to allow diffusion of adequate nutrients and gases to cells in said micro-organ explants and diffusion of cellular waste out of said micro-organ explants so as to minimize cellular toxicity and concomitant death due to insufficient nutrition and accumulation of said waste in said micro-organ explants;
(c) genetically modifying said plurality of micro-organ explants, so as to obtain a plurality of genetically modified micro-organ explants having at least a portion of their cells expressing and secreting said proteins differing by said at least one amino acid;
(d) implanting said plurality of genetically modified micro-organ explants within a plurality of recipient subjects; and
(e) comparatively determining at least one pharmacological, physiological and/or therapeutic, quantitative or qualitative, parameters or effects of said proteins differing by said at least one amino acid in said recipient subject.

69. The method of claim 68, wherein said recombinant gene products are encoded by an expressed sequence tag (EST).

70. The method of claim 68, wherein said recombinant gene products are of an unknown function.

71. The method of claim 68, wherein said recombinant gene products are of a known function.

72. The method of claim 68, wherein said recombinant gene products are of a suspected function.

73. The method of claim 68, wherein said recombinant gene products are of a suspected function based on sequence similarity to a protein of a known function.

74. The method of claim 68, wherein each of said recombinant gene products is encoded by a polynucleotide having a modified nucleotide sequence as compared to a corresponding natural polynucleotide.

75. The method of claim 68, wherein said cells of said micro-organ explants expressing and secreting said recombinant gene products are a result of genetic modification of at least a portion of the population of cells by transfection with recombinant virus carrying recombinant genes encoding said recombinant gene products.

76. The method of claim 75, wherein said recombinant virus is selected from the group consisting of a recombinant hepatitis virus, a recombinant adenovirus, a recombinant adeno-associated virus, a recombinant papilloma virus, a recombinant retrovirus, a recombinant cytomegalovirus, a recombinant simian virus, a recombinant lenti virus and a recombinant herpes simplex virus.

77. The method of claim 68, wherein said cells of said micro-organ explants expressing and secreting said recombinant gene products are transduced with foreign nucleic acid sequences via a transduction method selected from the group consisting of calcium-phosphate mediated transfection, DEAE-dextran mediated transfection, electroporation, liposome-mediated transfection, direct injection, gene gun transduction, pressure enhanced uptake of DNA and receptor-mediated uptake.

78. The method of claim 68, wherein said cells of said micro-organ explants expressing and secreting said recombinant gene products are a result of genetic modification of at least a portion of the population of cells by uptake of a non-viral vectors carrying recombinant genes encoding said recombinant gene products.

79. The method of claim 78, wherein said cells are transduced with foreign nucleic acid sequences via a transduction method selected from the group consisting of calcium-phosphate mediated transfection, DEAE-dextran mediated transfection, electroporation, liposome-mediated transfection, direct injection, gene gun transduction, pressure enhanced uptake of DNA and receptor-mediated uptake.

80. The method of claim 68, wherein expression of said recombinant gene products is under a control of an inducible promoter.

81. The method of claim 80, wherein said cells of said micro-organ explant cease to express and secrete said recombinant gene products, following administration of a repressor agent.

82. The method of claim 68, wherein expression of said recombinant gene products is under a control of a constitutive promoter.

83. The method of claim 68, wherein said recombinant gene products are selected from the group consisting of recombinant proteins and recombinant functional RNA molecules.

84. The method of claim 68, wherein said recombinant gene products are normally produced by the organ from which the micro-organ explants are derived.

85. The method of claim 68, wherein said recombinant gene products are normally not produced by the organ from which the micro-organ explants are derived.

86. The method of claim 68, wherein said recombinant gene products are encoded with known tag peptide sequences to be inserted into the recombinant proteins.

87. The method of claim 68, wherein said recombinant gene products are encoded with polycistronic recombinant nucleic acids including IRES site sequences, sequences encoding reporter proteins, and sequences encoding the proteins of interest.

88. The method of claim 68, wherein said recombinant gene products comprise marker proteins.

89. The method of claim 68, wherein said recombinant gene products are selected from the group consisting of natural or non-natural insulins, amylases, proteases, lipases, kinases, phosphatases, glycosyl transferases, trypsinogens, chymotrypsinogens, carboxypeptidases, hormones, ribonucleases, deoxyribonucleases, triacylglycerol lipases, phospholipase A2, elastases, amylases, blood clotting factors, UDP glucuronyl transferases, ornithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietins, epidermal growth factors, hepatic erythropoietic factors (hepatopoietin), liver-cell growth factors, interleukins, interferons, negative growth factors, fibroblast growth factors, transforming growth factors of the α family, transforming growth factors of the β family, gastrins, secretins, cholecystokinins, somatostatins, substance P and transcription factors.

90. The method of claim 68, wherein said micro-organ explants are immune-protected by biocompatible immuno-protective sheaths.

91. The method of claim 68, wherein said at least one pharmacological, physiological and/or therapeutic effect comprises efficacy.

92. The method of claim 68, wherein said at least one pharmacological, physiological and/or therapeutic effect comprises toxicity.

93. The method of claim 68, wherein said at least one pharmacological, physiological and/or therapeutic effect comprises mutagenicity.

94. The method of claim 68, wherein said at least one pharmacological, physiological and/or therapeutic effect comprises carcinogenicity.

95. The method of claim 68, wherein said at least one pharmacological, physiological and/or therapeutic effect comprises teratogenicity.

96. The method of claim 68, wherein said recipient subject is an established animal model for a human disease.

97. The method of claim 68, wherein prior to said implanting, in vitro secretion levels of said gene products from said micro-organs are determined.

98. The method of claim 97, wherein prior to said step of implanting, in vitro secretion levels of said gene products from said micro-organs are determined and an in vitro-in vivo correlation model is constructed so as to obtain a predetermined expression level in said animal model.

99. The method of claim 68, used for comparatively determining in vivo effects of protein-based drugs.

100. The method of claim 68, used for comparatively analyzing at least one pharmacokinetic parameter of protein-based drugs for in vivo.

101. The method of claim 68, used for comparatively analyzing drug efficacies of protein-based drugs in vivo.

102. The method of claim 68, used for comparatively analyzing toxicities of protein-based drug in vivo.

103. The method of claim 68, used for comparatively analyzing mutagenicities of protein-based drug in vivo.

104. The method of claim 68, used for comparatively analyzing carcinogenicities of protein-based drug in vivo.

105. The method of claim 68, used for comparatively analyzing teratogenicities of protein-based drug in vivo.

106. The method claim 68, wherein said dimensions are selected such that cells positioned deepest within said micro-organ explants are at least about 125-150 micrometers and not more than about 225-250 micrometers away from a nearest surface of said micro-organ explants.

107. The method of claim 106, wherein said organ is selected from the group consisting of a lymph system organ, a pancreas, a liver, a gallbladder, a kidney, a digestive tract organ, a respiratory tract organ, a reproductive system organ, a skin, a urinary tract organ, a blood-associated organ, a thymus and a spleen.

108. The method of claim 106, wherein said micro-organ explants comprise epithelial and connective tissue cells, arranged in a microarchitecture similar to the microarchitecture of the organ from which the explants were obtained.

109. The method of claim 106, wherein the organ is pancreas and the populations of cells comprise islets of Langerhan.

110. The method of claim 106, wherein the organ is skin and the explants comprise at least one hair follicle and at least one gland.

111. The method of claim 106, wherein the organ is a diseased tissue, and the explants comprise populations of hyperproliferative or neoproliferative cells from the diseased tissue.

112. The method of claim 68, wherein each of said micro-organ explants has a surface area to volume index characterized by the formula 1/x+1/a>1.5 mm-1; wherein ‘x’ is a tissue thickness and ‘a’ is a width of said tissues in millimeters.

113. The method of claim 112, wherein said organ is selected from the group consisting of a lymph system organ, a pancreas, a liver, a gallbladder, a kidney, a digestive tract organ, a respiratory tract organ, a reproductive system organ, a skin, a urinary tract organ, a blood-associated organ, a thymus and a spleen.

114. The method of claim 112, wherein said micro-organ explants comprise epithelial and connective tissue cells, arranged in a microarchitecture similar to the microarchitecture of the organ from which the explants were obtained.

115. The method of claim 112, wherein the organ is pancreas and the populations of cells comprise islets of Langerhan.

116. The method of claim 112, wherein the organ is skin and the explants comprise at least one hair follicle and at least one gland.

117. The method of claim 112, wherein the organ is a diseased tissue, and the explants comprise populations of hyperproliferative or neoproliferative cells from the diseased tissue.

118. The method of claim 68, wherein said micro-organ explants are derived from the recipient subjects.

119. The method of claim 68, wherein said donor subject is a human being.

120. The method of claim 68, wherein said donor subject is a non-human animal.

121. The method of claim 68, wherein said recipient subjects are human beings.

122. The method of claim 68, wherein said recipient subjects are non-human animals.

123. The method of claim 68, wherein said cells of said micro-organ explants expressing and secreting said recombinant gene products do so in a continuous, sustained fashion.

124. The method of claim 68, wherein said cells of said micro-organ explant expressing and secreting said recombinant gene products do so in a continuous, sustained fashion, following administration of an inducing agent.

125. The method of claim 124, wherein said cells of said micro-organ explants cease to express and secrete said recombinant gene products, following removal of said inducing agent.

126. The method of claim 68, wherein comparatively determining said at least one quantitative or qualitative pharmacological, physiological and/or therapeutic parameters or effects of said recombinant gene products in said recipient subject comprises determining survival.

127. The method of claim 68, wherein comparatively determining said at least one quantitative or qualitative pharmacological, physiological and/or therapeutic parameters or effects of said recombinant gene products in said recipient subjects comprises protein-drug synergistic effects.

128. The method of claim 68, wherein comparatively determining said at least one quantitative or qualitative pharmacological, physiological and/or therapeutic parameters or effects of said recombinant gene products in said recipient subjects comprises protein-drug antagonistic effects.

129. The method of claim 68, wherein comparatively determining said at least one quantitative or qualitative pharmacological, physiological and/or therapeutic, parameters or effects of said recombinant gene products in said recipient subjects comprises determining pathogen burden within at least one organ.

130. The method of claim 68, wherein comparatively determining said at least one quantitative or qualitative pharmacological, physiological and/or therapeutic, parameters or effects of said recombinant gene products in said recipient subjects comprises using at least one of the following assays: ELISA, Western blot analysis, HPLC, mass spectroscopy, GLC, immunohistochemistry, RIA, metabolic studies, patch-clamp analysis, perfusion assays, PCR, RT-PCR, Northern blot analysis, Southern blot analysis, RFLP analysis, nuclear run-on assays, gene mapping, cell proliferation assays and cell death assays.

131. A method of determining functional relations between recombinant gene products in vivo, the method comprising:
(a) providing at least one first polynucleotide encoding a first recombinant gene product;
(b) providing at least one second polynucleotide encoding a second recombinant gene product whose expression potentially functionally modifies or regulates the expression and/or function of said first recombinant gene product;
(c) obtaining a plurality of micro-organ explants from a donor subject, each of said plurality of micro-organ explants comprising a population of cells, each of said plurality of micro-organ explants maintaining a microarchitecture of an organ from which it is derived and at the same time having dimensions selected so as to allow diffusion of adequate nutrients and gases to cells in said micro-organ explants and diffusion of cellular waste out of said micro-organ explants so as to minimize cellular toxicity and concomitant death due to insufficient nutrition and accumulation of said waste in said micro-organ explants;
(d) genetically modifying said plurality of micro-organ explants, so as to obtain a plurality of genetically modified micro-organ explants having at least some of their cells expressing and secreting said first and/or second recombinant gene products;
(e) implanting said plurality of genetically modified micro-organ explants within a plurality of recipient subjects; and
(f) determining said functional relations between said first and second recombinant gene products in vivo.

132. The method of claim 131, wherein said recombinant gene products are encoded by expressed sequence tags (ESTs).

133. The method of claim 131, wherein said recombinant gene products are of an unknown function.

134. The method of claim 131, wherein said recombinant gene products are of a known function.

135. The method of claim 131, wherein said recombinant gene products are of a suspected function.

136. The method of claim 131, wherein said recombinant gene products are of a suspected function based on sequence similarity to a protein of a known function.

137. The method of claim 131, wherein said recombinant gene products are encoded by polynucleotides having modified nucleotide sequences as compared to a corresponding natural polynucleotide.

138. The method of claim 131, wherein said cells of said micro-organ explants expressing and secreting said recombinant gene products are a result of genetic modification of at least a portion of the population of cells by transfection with a recombinant virus carrying a recombinant gene encoding said recombinant gene products.

139. The method of claim 138, wherein said recombinant virus is selected from the group consisting of a recombinant hepatitis virus, a recombinant adenovirus, a recombinant adeno-associated virus, a recombinant papilloma virus, a recombinant retrovirus, a recombinant cytomegalovirus, a recombinant simian virus, a recombinant lenti virus and a recombinant herpes simplex virus.

140. The method of claim 131, wherein said cells of said micro-organ explants expressing and secreting said recombinant gene products are transduced with a foreign nucleic acid sequence via a transduction method selected from the group consisting of calcium-phosphate mediated transfection, DEAE-dextran mediated transfection, electroporation, liposome-mediated transfection, direct injection, gene gun transduction, pressure enhanced uptake of DNA and receptor-mediated uptake.

141. The method of claim 131, wherein said cells of said micro-organ explants expressing and secreting said recombinant gene products are a result of genetic modification of at least a portion of the population of cells by uptake of non-viral vectors carrying recombinant genes encoding said recombinant gene products.

142. The method of claim 141, wherein said cells are transduced with foreign nucleic acid sequences via a transduction method selected from the group consisting of calcium-phosphate mediated transfection, DEAE-dextran mediated transfection, electroporation, liposome-mediated transfection, direct injection, gene gun transduction, pressure enhanced uptake of DNA and receptor-mediated uptake.

143. The method of claim 131, wherein said recombinant gene products are under a control of inducible promoters.

144. The method of claim 131, wherein said recombinant gene products are under a control of constitutive promoters.

145. The method of claim 131, wherein said at recombinant gene products are selected from the group consisting of recombinant proteins and recombinant functional RNA molecules.

146. The method of claim 131, wherein said recombinant gene products are normally produced by the organ from which the micro-organ explants are derived.

147. The method of claim 131, wherein said recombinant proteins are normally not produced by the organ from which the micro-organ explants are derived.

148. The method of claim 131, wherein said recombinant gene products are encoded with known tag peptide sequences to be inserted into the recombinant proteins.

149. The method of claim 131, wherein said recombinant gene products are encoded with polycistronic recombinant nucleic acids including IRES site sequences, sequences encoding reporter proteins, and sequences encoding the proteins of interest.

150. The method of claim 131, wherein said recombinant gene products comprise marker proteins.

151. The method of claim 131, wherein said recombinant gene products are selected from the group consisting of insulin, amylase, proteases, lipases, kinases, phosphatases, glycosyl transferases, trypsinogen, chymotrypsinogen, carboxypeptidases, hormones, ribonucleases, deoxyribonucleases, triacylglycerol lipases, phospholipase A2, elastases, amylases, blood clotting factors, UDP glucuronyl transferases, ornithine transcarbamoylases, cytochrome p450 enzyme, adenosine deaminases, serum thymic factors, thymic humoral factors, thymopoietin, growth hormone, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the α family, a transforming growth factors of the β family, gastrin, secretin, cholecystokinin, somatostatin, serotinin, substance P and transcription factors.

152. The method of claim 131, wherein said micro-organ explants are immune-protected by biocompatible immuno-protective sheaths.

153. The method of claim 131, wherein determining functional relations between said recombinant gene products comprises determining a level of RNA expression of said first recombinant gene product in a presence and in an absence of said second gene product.

154. The method of claim 131, wherein determining functional relations between said recombinant gene products comprises determining a level of protein expression of said first recombinant gene product in a presence and in an absence of said second gene product.

155. The method of claim 131, wherein determining functional relations between said recombinant gene products comprises determining a level of activity of said first recombinant gene product in a presence and in an absence of said second gene product.

156. The method of claim 131, wherein determining functional relations between said recombinant gene products comprises determining at least one pharmacological, physiological and/or therapeutic parameter or effect of at least one of said gene-products.

157. The method of claim 156, wherein at least one pharmacological, physiological and/or therapeutic effect comprises efficacy.

158. The method of claim 156, wherein said at least one pharmacological, physiological and/or therapeutic effect comprises toxicity.

159. The method of claim 156, wherein said at least one pharmacological, physiological and/or therapeutic effect comprises mutagenicity.

160. The method of claim 156, wherein said at least one pharmacological, physiological and/or therapeutic effect comprises carcinogenicity.

161. The method of claim 156, wherein said at least one pharmacological, physiological and/or therapeutic effect comprises teratogenicity.

162. The method of claim 156, wherein said at least one pharmacological, physiological and/or therapeutic effect comprises determining survival.

163. The method of claim 156, wherein said at least one pharmacological, physiological and/or therapeutic parameter or effect comprises determining pathogen burden within at least one organ.

164. The method of claim 131, wherein determining functional relations between said recombinant gene products employs at least one of the following assays: ELISA, Western blot analysis, HPLC, mass spectroscopy, GLC, immunohistochemistry, RIA, metabolic studies, patch-clamp analysis, perfusion assays, PCR, RT-PCR, Northern blot analysis, Southern blot analysis, RFLP analysis, nuclear run-on assays, gene mapping, cell proliferation assays and cell death assays.

165. The method of claim 156, wherein said at least pharmacological, physiological and/or therapeutic parameter or effect is determined in a qualitative or quantitative manner.

166. The method of claim 131, wherein said functional relations between said recombinant gene products comprise direct effects of one recombinant gene product on another.

167. The method of claim 166, wherein said direct effects comprise functional and/or structural modification of a recombinant gene product.

168. The method of claim 167, wherein said functional and/or structural modification comprises cleavage, phosphorylation, glycosylation, methylation or assembly of a recombinant gene product.

169. The method of claim 168, wherein said functional and/or structural modification comprises processing of a recombinant gene product to its active form.

170. The method of claim 131, wherein said functional relations between said recombinant gene products comprise indirect effects of one recombinant gene product on another.

171. The method of claim 170, wherein said indirect effects comprise functional and/or structural modification of a recombinant gene product.

172. The method of claim 171, wherein said functional and/or structural modification comprises positive or negative effects on promoter sequences.

173. The method of claim 172, wherein said positive or negative effects on promoter sequences are mediated in trans.

174. The method of claim 131, wherein said recipient subject is an established animal model for a human disease.

175. The method of claim 131, wherein prior to said implanting, in vitro secretion levels of said gene products are determined.

176. The method of claim 174, wherein prior to said step of implanting, in vitro secretion levels of said gene products from said micro-organs are determined and an in vitro-in vivo correlation model is constructed for said animal model so as to enable quantitative prediction and adjustment of the expression levels in said animal model.

177. The method of claim 131, wherein determining said functional relations between said recombinant gene products comprises determining in vivo effects of at least one protein-based drug.

178. The method of claim 131, wherein determining said functional relations between said recombinant gene products comprises analyzing at least one pharmacokinetic parameter for at least one protein-based drug in vivo.

179. The method of claim 131, wherein determining said functional relations between said recombinant gene products comprises determining efficacy for at least one protein-based drug in vivo.

180. The method of claim 131, wherein determining said functional relations between said recombinant gene products comprises determining toxicity for at least one protein-based drug in vivo.

181. The method of claim 131, wherein determining said functional relations between said recombinant gene products comprises determining mutagenicity for at least one protein-based drug in vivo.

182. The method of claim 131, wherein determining said functional relations between said recombinant gene products comprises determining carcinogenicity for at least one protein-based drug in vivo.

183. The method of claim 131, wherein determining said functional relations between said recombinant gene products comprises determining teratogenicity for at least one protein-based drug in vivo.

184. The method claim 131, wherein said dimensions are selected such that cells positioned deepest within said micro-organ explants are at least about 125-150 micrometers and not more than about 225-250 micrometers away from a nearest surface of said micro-organ explants.

185. The method of claim 184, wherein said organ is selected from the group consisting of a lymph system organ, a pancreas, a liver, a gallbladder, a kidney, a digestive tract organ, a respiratory tract organ, a reproductive system organ, a skin, a urinary tract organ, a blood-associated organ, a thymus and a spleen.

186. The method of claim 184, wherein each of said micro-organ explants comprises epithelial and connective tissue cells, arranged in a microarchitecture similar to the microarchitecture of the organ from which the explants were obtained.

187. The method of claim 184, wherein the organ is pancreas and the populations of cells comprise islets of Langerhan.

188. The method of claim 184, wherein the organ is skin and the explants comprise at least one hair follicle and at least one gland.

189. The method of claim 184, wherein the organ is a diseased tissue, and the explants comprise populations of hyperproliferative or neoproliferative cells from the diseased tissue.

190. The method of claim 131, wherein each of said micro-organ explants has a surface area to volume index characterized by the formula 1/x+1/a>1.5 mm-1; wherein ‘x’ is a tissue thickness and ‘a’ is a width of said tissue in millimeters.

191. The method of claim 190, wherein said organ is selected from the group consisting of lymph system organs, pancreas, liver, gallbladder, kidney, digestive tract organs, respiratory tract organs, reproductive system organs, skin, urinary tract organs, blood-associated organs, thymus and spleen.

192. The method of claim 190, wherein each of said micro-organ explants comprises epithelial and connective tissue cells, arranged in a microarchitecture similar to the microarchitecture of the organ from which the explants were obtained.

193. The method of claim 190, wherein the organ is a pancreas and the populations of cells comprise islets of Langerhan.

194. The method of claim 190, wherein the organ is skin and the explants comprise at least one hair follicle and at least one gland.

195. The method of claim 190, wherein the organ is a diseased tissue, and the explants comprise population of hyperproliferative or neoproliferative cells from the diseased tissue.

196. The method of claim 131, wherein said micro-organ explants are derived from the recipient subject.

197. The method of claim 131, wherein said donor subject is a human being.

198. The method of claim 131, wherein said donor subject is a non-human animal.

199. The method of claim 131, wherein said recipient is a human being.

200. The method of claim 131, wherein said recipient subject is a non-human animal.

201. The method of claim 131, wherein said cells of said micro-organ explants express and secrete said recombinant gene products in a continuous, sustained fashion.

202. The method of claim 131, wherein said cells of said micro-organ explants express and secrete said recombinant gene products in a continuous, sustained fashion, following administration of an inducing agent.

203. The method of claim 195, wherein said cells of said micro-organ explants cease to express and secrete said recombinant gene products, following removal of said inducing agent.
